SEQUENCE LISTING (4 sequences)

SEQ ID NO: 1 - protein, 1368 aa, Streptococcus pyogenes [sequence omitted]

SEQ ID NO: 2 - protein, 1053 aa, Staphylococcus auricularis
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 4 - protein, 1306 aa, Acidaminococcus sp.
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

(19) United States
(12) Patent Application Publication
(10) Pub. No.: US 2025/0375508 A1
(43) Pub. Date: Dec. 11, 2025

(54) METHODS AND COMPOSITIONS FOR CAS IMMUNE TOLERANCE INDUCTION TO SUPPORT CRISPR-CAS IN VIVO GENE EDITING

(71) Applicants: University of Pittsburgh - Of the Commonwealth System of Higher Education, Pittsburgh, PA (US); The Regents of the University of California, Oakland, CA (US)

(72) Inventors: Stephen C. Balmert, Cranberry Township, PA (US); Louis D. Falo, Wexford, PA (US); Steven R. Little, Pittsburgh, PA (US); David H. Raulet, Berkley, CA (US); Gregory M. Barton, Piedmont, CA (US); Emrullah Korkmaz, Pittsburgh, PA (US)

(73) Assignees: University of Pittsburgh - Of the Commonwealth System of Higher Education, Pittsburgh, PA (US); The Regents of the University of California, Oakland, CA (US)

(21) Appl. No.: 18/877,079

(22) PCT Filed: Jun. 28, 2023
(86) PCT No.: PCT/US2023/069282

§ 371 Date: Dec. 19, 2024

Related U.S. Application Data
(60) Provisional application No. 63/356,932, filed on Jun. 29, 2022.

(51) Int. Cl. A61K 39/00; A61K 9/00; A61K 31/203; A61K 39/39; A61K 48/00; A61P 37/06

(52) U.S. Cl. CPC A61K 39/001134; A61K 9/0021; A61K 31/203; A61K 39/001; A61K 39/00114; A61K 39/001154; A61K 39/39; A61K 48/0025; A61K 48/005; A61P 37/06; A61K 2039/54; A61K 2039/55533; A61K 2039/55555

(57) ABSTRACT

Tolerogenic compositions are disclosed that are of use for inducing a tolerogenic immune response to a CRISPR-Cas effector polypeptide in a subject. In some aspects, the tolerogenic composition includes: a) one or more microparticles; b) one or more regulatory T cell (Treg) stimulating agents encapsulated within each microparticle; and c) a CRISPR-Cas effector polypeptide or immunogenic fragment thereof, or a fusion polypeptide comprising a CRISPR-Cas effector polypeptide or immunogenic fragment thereof. In other aspects, the tolerogenic composition includes a) a dissolvable microneedle array; b) one or more agents that promote differentiation of tolerogenic DCs in the dissolvable microneedle array; and c) a CRISPR-Cas effector polypeptide or immunogenic fragment thereof, or a fusion polypeptide comprising a CRISPR-Cas effector polypeptide or immunogenic fragment thereof.

### CROSS REFERENCE TO RELATED APPLICATIONS

This claims the benefit of U.S. Provisional Application No. 63/356,932, filed Jun. 29, 2022, which is incorporated herein by reference.

### STATEMENT OF GOVERNMENT SUPPORT

This invention was made with government support under grant no. AR074285, awarded by the National Institutes of Health (NIH), and grant no. HR0011-17-2-0043, awarded by the Defense Advanced Research Projects Agency (DARPA). The government has certain rights in the invention.

### FIELD OF THE DISCLOSURE

This relates to the field of gene editing, specifically to methods for inducing immune tolerance to a CRISPR-Cas effector polypeptide.

### REFERENCE TO AN ELECTRONIC SEQUENCE LISTING

The contents of the electronic sequence listing, named Sequences.xml, having a size of 12,288 bytes, and a Date of Creation: Jun. 27, 2023, is herein incorporated by reference in its entirety.

### BACKGROUND

Gene editing is being increasingly used in therapeutic settings. To edit genes in cells in vivo, a Clustered Regularly Interspaced Short Palindromic Repeat (CRISPR)-Cas effector protein, such as Cas9, and a guide RNA, are contacted with a target nucleic acid in the cells, where the CRISPR-Cas effector polypeptide and guide RNA together modify the target nucleic acid or its expression. However, the CRISPR-Cas effector polypeptide is foreign to a mammalian host such as a human and may elicit an immune response. Induced antibodies and/or T cells specific for the CRISPR-Cas effector polypeptide may neutralize the CRISPR-Cas effector polypeptide and/or kill cells that include the CRISPR-Cas effector polypeptide. Such an immune response could reduce the efficiency of gene editing and preclude future editing using the CRISPR-Cas effector polypeptide. A need remains for methods that elicit immune tolerance to a CRISPR-Cas effector polypeptide, such as, but not limited to, Cas9.

### SUMMARY OF THE DISCLOSURE

Tolerogenic compositions are disclosed that are of use for inducing a tolerogenic immune response to a CRISPR-Cas effector polypeptide in a subject. In some embodiments, the tolerogenic composition includes: a) a microparticle; b) one or more regulatory T cell (Treg) stimulating agents encapsulated within the microparticle; and c) a CRISPR-Cas effector polypeptide or immunogenic fragment thereof, or a fusion polypeptide comprising a CRISPR-Cas effector polypeptide or the immunogenic fragment thereof. In other embodiments, the tolerogenic composition includes a) a dissolvable microneedle array; b) one or more agents that promote differentiation of tolerogenic DCs in the dissolvable microneedle array; and c) a CRISPR-Cas effector polypeptide or immunogenic fragment thereof, or a fusion polypeptide comprising a CRISPR-Cas effector polypeptide or immunogenic fragment thereof.

The foregoing and other features and advantages of the invention will become more apparent from the following detailed description of several embodiments which proceeds with reference to the accompanying figures.

### BRIEF DESCRIPTION OF THE FIGURES

FIGS. 1A-1C. Schematic. FIG. 1A Antigens delivered alone or with adjuvants can promote innate inflammation, which triggers migration of pro-inflammatory DCs to skin draining lymph nodes (DLN) and differentiation of inflammatory effector T cells (Teff). FIG. 1B TRI MPs co-delivered subcutaneously with protein antigen (e.g., Cas9) condition the skin DLN microenvironment to promote regulatory T-cell (Treg) differentiation. FIG. 1C Co-delivery of antigen (e.g., Cas9) with MC903 (a vitamin D analog) microneedle arrays (MNAs) conditions the skin microenvironment and provides tolerogenic context for DCs that take up antigen. migrate to DLN, and induce preferential Treg differentiation and/or reduce differentiation of Teff.

FIG. 2: Immunization with Cas9 expressing cells elicits Cas9-specific CD8⁺ T cells. C57BL/6 mice were immunized with 1×10⁷ LPS Cas9-Tg splenocytes via intra-peritoneal injection (red circles) or subcutaneous injection (open squares) on day 0 and day 14. On day 26 splenocytes from immunized mice were harvested. Cells were incubated with control cells (B6) or cells expressing Cas9 antigen (Cas9) that were previously activated with LPS treatment (+) or not (−). Shown is intracellular cytokine staining quantifying the frequency of CD8⁺ T cells producing IFNγ in response to re-stimulation (unpaired t-test. *p<0.05, **p<0.01).

FIG. 3: Immunization with Cas9 formulated with Adjuvant elicits Cas9-specific CD4⁺ T cells. C57BL/6 mice were immunized with 100 μg SpCas9 or PBS buffer formulated with TITERMAX® Gold adjuvant on day 0 and day 14. On day 21, splenocytes from mice immunized with PBS (left) or SpCas9 (right) were harvested and re-stimulated with Cas9 protein. Shown is the frequency of CD4+ T cells producing IFNγ as assessed by intracellular cytokine staining (2 way ANOVA, UN=unstimulated).

FIGS. 4A-4C: Treg-inducing microparticles with Cas9 expands splenic Tregs and reduces Cas9 specific CD4 T cell responses. C57BL/6 mice were pre-treated on day −12 with PBS buffer, blank microparticles (MP), or Treg-inducing microparticles (TRI-MP) with or without Cas9. Mice received one (FIGS. 4A-4B) or two (FIG. 4C) immunizations of 100 μg Cas9 protein formulated with TiterMax Gold adjuvant. Mice were euthanized 7 days post final immunization and splenic T cell responses were evaluated by IFNγ ELISPOT (FIG. 4A). flow cytometry (FIG. 4B), or cytokine bead array (FIG. 4C). IFNγ responses elicited by stimulation with medium (open circle), OVA (diamond), or Cas9 protein (squares) (FIG. 4A). Statistical comparisons with 2-way ANOVA with Tukey's multiple comparisons test. FIG. 4B: Percentages of regulatory T cells in the spleen. Ordinary one-way ANOVA, **p<0.01, ***p<0.001, ****p<0.0001. FIG. 4C: Cytokine bead array quantifying secreted IFNγ following a 3-day stimulation, 2-way ANOVA with Tukey's multiple comparisons test.

FIG. 5: Tolerogenic microneedle arrays reduce Cas9 Specific CD4 T Cell responses. C57BL/6 mice were left untreated (far left) or received intra-dermal applications of Cas9 alone or MNAs containing Cas9 (50 μg) or BSA (50 μg) on days 0, 3, and 6. On day 13. mice were immunized with Cas9 formulated with TITERMAX® Gold at a distal site. Seven days after the challenge, splenocytes were harvested and stimulated with medium (open circles) or Cas9 protein (closed squares). Shown are the frequencies of CD4 T cells secreting IFNγ by intracellular cytokine staining (2-way ANOVA with Tukey's multiple comparisons test).

FIG. 6: SpCas9-specific delayed type hypersensitivity (DTH) assay. Mice were sensitized (immunized) to S. pyogenes Cas9 (SpCas9) antigen either by application of a Cas9 (antigen)+Poly(I:C) (adjuvant) MNA, or by subcutaneous injection of Cas9-expressing splenocytes. Seven days later, mice were challenged (re-exposed to Cas9 antigen) at the right ear via Cas9 MNA, and ear thickness was measured daily over the next four days. A blank (empty) MNA was applied to the contralateral ear to control for any increase in ear thickness not related to the Cas9 antigen-specific inflammatory response. Ear swelling associated T-cell mediated DTH response to Cas9 was reported as changes (delta) in ear thickness (i.e., Cas9 MNA treated right ear−Blank MNA treated left ear). Greater ear swelling is consistent with a stronger Cas9-specific, T-cell mediated immune response.

FIG. 7: Prophylactic tolerance inducing with Cas9 and tolerogenic vitamin D3 analog (MC903) microneedle arrays. Prior to sensitization with Cas9+PolyIC MNAs, mice were treated by three consecutive applications of Cas9+MC903 MNAs (days 0, 3, and 6). Three days later (day 9), mice were sensitized to Cas9 antigen by application of a Cas9+Poly(I:C) MNA. Seven days later, mice were challenged at the right ear via Cas9 MNA, and ear thickness was measured daily over the next four days. A blank MNA was applied to the contralateral ear to control for any increase in ear thickness not related to the Cas9 antigen-specific inflammatory response. Ear swelling associated with a T-cell mediated DTH response to Cas9 was reported as changes (delta) in ear thickness (i.e., Cas9 MNA treated right ear−Blank MNA treated left ear). Pre-treatment with Cas9+MC903 MNAs (prophylactic tolerization) reduced subsequent sensitization, resulting in less DTH (car swelling) response.

FIGS. 8A-8C. Prophylactic tolerance inducing with Cas9 and MC903 microneedle arrays. Four days after ear challenge (as in FIG. 7), ear skin draining lymph nodes (DLN) were isolated and T-cell responses evaluated by flow cytometry. FIGS. 8A-8B: Total cells per ear DLN: (1) CD4+FoxP3+ regulatory T cells (Treg), (2) CD4+ T-bet+type 1 helper T cells (Th1), (3) CD8+ T-bet+type 1 cytotoxic T cells (Tc1), (4) Treg/Th1 ratio, (5) Treg/(Th1+Tc1) or Treg (effector T cell, Teff) ratio. FIG. 8C: Frequencies of FoxP3+ Treg, T-bet+ Th1, and T-bet+ Tc1 populations in ear DLN. Groups were compared by one-way independent ANOVA, followed by Tukey's multiple comparisons test. Compact letter display indicates significant differences (p<0.05, different letters) or non-significant differences (p>0.05, common letters). As a non-limiting example, a group labeled “a” is significantly different than a group labeled “b,” but not different than another group labeled a or group labeled “ab.” A group labeled “ab” is not significantly different than a group labeled “a” or group labeled “b.” but is significantly different than a group labeled “c.”

FIGS. 9A-9B: Therapeutic desensitization with Cas9+Mc903 MNAs. Tolerance was induced by treatment of previously sensitized mice (i.e., mice with pre-existing immunity to Cas9) with Cas9+MC903 MNAs. In the therapeutic desensitization model, mice were first sensitized to SpCas9 by subcutaneous injection of SpCas9-expressing splenocytes. Starting one week later, some mice were treated with Cas9+MC903 MNAs (days 7, 10, and 13). Five days after the last treatment with Cas9+MC903 MNAs (day 18), all mice received an adoptive transfer of a mix of Cas9-expressing CFSE^{high} “target” cells and “control” CFSE^{low} cells (i.e., wild-type splenocytes that don't express Cas9). The next day, spleens of mice were processed, and analyzed by flow cytometry (FIG. 9A) to measure specific cell lysis (i.e., specific killing of Cas9-expressing target cells by Cas9-specific effector T cells, especially cytotoxic T cells). Percent specific lysis is calculated as {1-[(mean CFSE^{low}/CFSE^{high} ratio from naïve mice)/(CFSE^{low}/CFSE^{high} ratio from immunized mouse)]}×100% (FIG. 9B). Sensitized and Desensitized groups were compared by independent t-test.

FIG. 10: SpCas9 release from alginate hydrogel microparticles. Formulations: SpCas9 (2.5 mg)+1% Alginate (med viscosity, 100 mg alginate)—labeled “b”; SpCas9 (2.5 mg)+1% Alginate (low viscosity, 100 mg alginate)—labeled “a.”

FIGS. 11A-11D. Sequences of CRISPR-Cas effector polypeptides. SEQ ID NOs: 1-4 are shown.

FIG. 12. Pre-treatment with SpCas9+MC903 MNAs reduces sensitization and subsequent delayed type hypersensitivity (DTH) response. A line graph showing the change in ear thickness for each of the listed treatments, and representative digital images from the control and the animals treated with SpCas9 and MC903 mRNA are shown.

### DETAILED DESCRIPTION OF SEVERAL EMBODIMENTS

Experiments were conducted to produce a reduced immune response to a CRISPR-Cas effector polypeptide, such as Cas9 and other editors, in order to facilitate in vivo editing without the induction of an immune response. The reduction of an immune response, such as an anti-Cas9 T cell or antibody response, has at least two applications: i) improvement of the efficiency of an initial editing procedure, which often involves multiple rounds of editing with the same guide RNA, in the course of which immune responses to the editor may be induced leading to the death of cells undergoing editing and/or neutralization of the editor before it reaches the cells in which editing is to occur; (ii) enablement of patients that had previously undergone an editing procedure to receive a later procedure to edit different genes. Tolerogenic compositions are disclosed herein that are of use for inducing a tolerogenic immune response to a CRISPR-Cas effector polypeptide in a subject. In some embodiments, the tolerogenic composition includes: a) a microparticle; b) one or more regulatory T cell (Treg) stimulating agents encapsulated within the microparticle; and c) a CRISPR-Cas effector polypeptide or immunogenic fragment thereof, or a fusion polypeptide comprising a CRISPR-Cas effector polypeptide or immunogenic fragment. In other embodiments, the tolerogenic composition includes a) a dissolvable microneedle array; b) a vitamin D analog. such as, but not limited to, MC903; and c) a CRISPR-Cas effector polypeptide or immunogenic fragment thereof, or a fusion polypeptide comprising a CRISPR-Cas effector polypeptide or immunogenic fragment thereof. In some embodiments, the CRISPR-Cas effector polypeptide or immunogenic fragment thereof, or a fusion polypeptide comprising a CRISPR-Cas effector polypeptide, is co-administered with one or more additional agents.

### Terms

Unless otherwise noted, technical terms are used according to conventional usage. Definitions of common terms in molecular biology can be found in Krebs et al (Eds.), Lewin's Genes XII, published by Jones & Bartlett Publishers, 2017; and Meyers et al. (Eds.), The Encyclopedia of Cell Biology und Molecular Medicine, published by Wiley-VCH in 16 volumes, 2008; and other similar references.

As used herein, the singular forms “a,” “an,” and “the” refer to both the singular as well as plural unless the context clearly indicates otherwise. Further, “or” also include “and/or”; thus, “a first particle or a second particle” also includes “a first particle and/or a second particle,” and compositions of use in the methods herein can be used alone or in combination. Although many methods and materials similar or equivalent to those described herein can be used, particular suitable methods and materials are described below. The term “comprises” means “includes.” The term “about” means within five percent, unless otherwise indicated. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Administration: The introduction of a composition, such as a small molecule inhibitor, into a subject by a chosen route. Administration can be local or systemic. Exemplary routes of administration include, but are not limited to, oral, injection (such as subcutaneous, intramuscular, intradermal, intraperitoneal, intratumoral, intravenous), sublingual, rectal, transdermal (for example, topical), intranasal, vaginal, and inhalation routes.

Agent: A drug, medicament, pharmaceutical, therapeutic, nutraceutical, biological molecule, or other compound that may be administered to a subject to effect a change, such as treatment, amelioration, or prevention of a disease or disorder or at least one symptom associated therewith, or altering an immune response, including a regulatory immune response. An agent may be a “small molecule,” generally having a molecular weight of about 2000 daltons or less. The active agent may also be a “biological agent.” Biological agents include proteins, antibodies, antibody fragments, peptides, oligonucleotides and various derivatives of such materials.

Animal: Living multi-cellular vertebrate organisms, a category that includes, for example, mammals and birds. The term mammal includes both human and non-human mammals. Similarly, the term “subject” includes both human and veterinary subjects.

Cas9: An RNA-guided DNA endonuclease enzyme that can cut DNA. Cas9 has two active cutting sites (HNH and RuvC), one for each strand of a double helix. Catalytically inactive (deactivated) Cas9 (dCas9) is also encompassed by this disclosure. In some examples, a dCas9 includes one or more of the following point mutations: D10A. H840A, and N863A.

Cas9 nucleic acid and protein sequences are publicly available. For example. GenBank® Accession Nos. nucleotides 796693 . . . 800799 of CP012045.1 and nucleotides 1100046 . . . 1104152 of CP014139.1 disclose Cas9 nucleic acids, and GENBANK® Accession Nos. AMA70685.1 and AKP81606.1 disclose Cas9 proteins. In some examples, the Cas9 is a deactivated form of Cas9 (dCas9), such as one that is nuclease deficient (e.g., those shown in GENBANK® Accession Nos. AKA60242.1 and KR011748.1). In certain examples, Cas9 has at least 80% sequence identity, for example at least 85%, at least 90%, at least 95%, at least 96%, at least 97%, at least 98%, or at least 99% sequence identity to such sequences, and retains the ability to cut DNA. Cas9 mini proteins are also included.

Caspase: An enzyme that is that a cysteine-aspartic protease, cysteine aspartase or cysteine-dependent aspartate-directed protease. Caspases are a family of protease enzymes playing essential roles in programmed cell death. They are named caspases due to their specific cysteine protease activity, wherein a cysteine in its active site nucleophilically attacks and cleaves a target protein only after an aspartic acid residue.

Conservative variants: “Conservative” amino acid substitutions are those substitutions that do not substantially affect or decrease an activity of a polypeptide. Specific, non-limiting examples of a conservative substitution include the following examples:

| Original | Conservative |
|---|---|
| Residue | Substitutions |
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variation also includes the use of a substituted amino acid in place of an unsubstituted parent amino acid, provided that the polypeptide binds with the same affinity as the unsubstituted (parental) polypeptide. Non-conservative substitutions are those that alter the activity of the polypeptide.

Co-Administration: The administration of an agent disclosed herein with at least one other therapeutic or diagnostic agent within the same general time period, and does not require administration at the same exact moment in time (although co-administration is inclusive of administering at the same exact moment in time). Thus, co-administration may be simultaneous or within a specified time frame. In certain embodiments, a plurality of therapeutic and/or diagnostic agents may be co-administered by encapsulating the agents within the microparticles or using the microneedles disclosed herein.

Control: A reference standard. In some embodiments, the control is a negative control sample obtained from a healthy patient, or a subject treated with a carrier, non-targeted nucleic acid sequences, scrambled nucleic acid/amino acid sequences or untreated cells from a healthy patient. In other embodiments, the control is a positive control sample obtained from a patient that has been treated with an active agent. In still other embodiments, the control is a historical control or standard reference value or range of values (such as a previously tested control sample, such as a group of patients with known prognosis or outcome, or group of samples that represent baseline or normal values).

A difference, such as for an immune response, or in an assay (e.g. an ELISA, a RNA expression profile and the like) performed on a test sample and a control, can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%. at least about 100%, at least about 150%, at least about 200%, at least about 250%, at least about 300%, at least about 350%, at least about 400%, at least about 500%, or greater than 500%.

CRISPR (Clustered Regularly InterSpaced Short Palindromic Repeats)/Cas (CRISPR-associated protein) Editing System: An engineered nuclease system based on a bacterial system that is used for genome engineering. It is based in part on the adaptive immune response of many bacteria and archaea. Such methods can be used to allow genetic material to be added, removed, or altered at particular locations, for example in a target DNA or RNA sequence. Thus, CRISPR/Cas systems can be used for nucleic acid targeting (such as DNA or RNA), for example to detect a target DNA or RNA, modify a target DNA or RNA at any desired location, or cut the target DNA or RNA at any desired location. Thus, such methods can be used to modify expression of a protein, for example by introducing a mutation to silene expression, such as knocking out the gene.

In one example, the method edits DNA, such as a genome, and uses a “CRISPR-Cas effector polypeptide.” The CRISPR-Cas effector polypeptide is an enzyme that cleaves the nucleic acid in this system. CRISPR-Cas effector polypeptides include, but are not limited to, Cas9. A CRISPR/Cas system can be engineered to create a break in DNA at a desired target in a genome of a cell, and harness the cell's endogenous mechanisms to repair the induced break by homology-directed repair (HDR) or nonhomologous end-joining (NHEJ). In another example, the CRISPR-Cas effector polypeptide breaks RNA, such as Cas13d nuclease (see for example WO 2019/040664). CRISPR-Cas effector polypeptide can be a type II, type V, or a type IV CRISPR-Cas effector polypeptide. Non-limiting examples of CRISPR-Cas effector polypeptides include Cas1, Cas1B, Cas2. Cas3. Cas4, Cas5, Cas6, Cas7, Cas8, Cas9 (also known as Csn1 and Csx12). Cas12, Cas10, Cas13, Cpf1, C2c3, C2c2 and C2c1Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1. Csx15, Csf1, Csf2, Csf3, Csf4,, Cas Lambda, CasX, CasY, and Cas phi, as well as homologs thereof.

Dendritic cells (DCs): Tolerogenic DCs are commonly defined by including low or intermediate levels of MHC II, costimulatory molecules CD80, CD86 and CD40, and chemokine receptor CCR7, in addition to an increased antigen uptake capacity. Tolerogenic DCs can express high levels of inhibitory molecules such as Ig-like transcripts (ILT) molecules (ILT3/ILT4) and/or PD-L molecules (PD-L1, PD-L2). Additionally, tolerogenic DCs can secrete low amounts of proinflammatory cytokines (IL-12p70) and high quantities of anti-inflammatory cytokines, such as IL-10. Tolerogenic DCs function to induce T cell anergy, T cell suppression and the generation of regulatory T cells by several mechanisms, including conversion of naive T cells into Tregs, release of immunosuppressive cytokines, and expression of functional indoleamine-2,3 dioxygenase (IDO).

Effective amount: An amount of agent, such as an immunogen, that is sufficient to elicit a desired response, such as a tolerogenic immune response in a subject. It is understood that the procedure used to obtain a tolerogenic immune response against an antigen of interest can require multiple administrations of a disclosed composition. Accordingly, an effective amount of a disclosed composition can be the amount of the immunogen sufficient to elicit a priming immune response in a subject that can be subsequently boosted with the same or a different composition to elicit a tolerogenic immune response.

Epitope: An antigenic determinant. These are particular chemical groups or peptide sequences on a molecule that are antigenic, such that they elicit a specific immune response, for example, an epitope is the region of an antigen to which B and/or T cells respond. Epitopes can be formed both from contiguous amino acids or noncontiguous amino acids juxtaposed by tertiary folding of a protein.

Forkhead box P3 (Foxp3): A transcription factor that regulates and orchestrates the molecular processes involved in Treg differentiation and function (Zheng and Rudensky, Nat. Immunol. 8:457-462, 2007). Treg cells are a type of T cell that have an important role in maintaining immune system homeostasis by suppressing over-reactive immune responses (Josefowicz et al. Annu. Rev. Immunol. 30, 531-564, 2012). Defects in Treg cells can lead to autoimmune disorders and immunopathology.

Conversely, certain tumors are enriched with Treg cells that suppress anti-tumor immune responses (Tanaka and Sakaguchi, Cell Res. 27, 109-118, 2017). Increased Foxp3 activity enhances Treg suppressor function, whereas decreased Foxp3 activity suppresses Treg suppressor function (Loo et al., Immunity 53, 143-157, 2020).

Immune Response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies. An immune response can be a “tolerogenic” immune response, and thus induce tolerance to a particular antigen. A tolerogenic immune response can include inducing and/or stimulate regulatory T cells (Treg) and/or tolerogenic dendritic cells. Tolerogenic dendritic cells induce tolerance through several mechanisms. Once stimulated, tolerogenic dendritic cells migrate to the draining lymph node and present antigens to T cells via interaction of MHC class II-antigen complexes on the dendritic cell with T cell receptors on the T cell. This can induce T cell clonal deletion, T cell anergy or the proliferation of Tregs. Collectively, these mechanisms produce tolerance to specific antigens. Thus, “immune tolerance” refers to a state of unresponsiveness of the immune system to substances, proteins, epitopes, or cells that would otherwise have the capacity to elicit an immune response in a given organism.

Isolated: An “isolated” biological component (e.g. nucleic acid, protein, or cell) has been substantially separated or purified away from other biological components in the environment (such as a cell or tissue) in which the component occurs, e.g., other chromosomal and extra-chromosomal DNA and RNA, proteins and cells. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Mammal: This term includes both human and non-human mammals. Similarly, the term “subject” includes both human and veterinary subjects.

Microneedle: Microscopic structures associated with an array, also referred to as a “microarray”, that are capable of piercing the stratum corneum to facilitate the transdermal or intradermal delivery of therapeutic agents or the sampling of fluids through the skin. The “array” refers to the medical devices described herein that include an ordered patten of one or more structures capable of piercing the stratum corneum to facilitate the transdermal delivery of therapeutic agents.

Microparticle: Microparticles generally refer to the general categories comprising liposomes, nanoparticles, microspheres, nanospheres, microcapsules, nanorod, and nanocapsules. A microparticle may be of composite construction and is not necessarily a pure substance; it may be spherical or any other shape.

In some cases, a microparticle includes one or more biodegradable polymers. The term “biodegradable.” as used herein, refers to the ability of materials to be broken down by normal chemical, biochemical and/or physical processes such as erosion, dissolution, corrosion, degradation, hydrolysis, abrasion, and their combinations. A microparticle can range in diameter between about 0.1 μm and about 1000 μm or any range therebetween. Additional information is provided below.

Modulate: To alter in a statistically significant manner. Modulation can be an increase or a decrease. One of skill in the art can identify an appropriate assay to determine a statistically significant increase or decrease in a parameter. These include, but are not limited to, a student's t-test or a paired ratio t test. Exemplary methods are provided in the Examples section.

Pharmaceutically Acceptable Carrier: Includes any and all solvents, dispersion media, coatings, antibacterial and antifungal agents, isotonic and absorption delaying agents, and the like, compatible with pharmaceutical administration (see, e.g., Remington: The Science and Practice of Pharmacy, The University of the Sciences in Philadelphia, Editor, Lippincott, Williams, & Wilkins, Philadelphia, PA,21^{st} Edition, 2005). Examples of such carriers or diluents include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Actual methods for preparing administrable compositions include those provided in Remington: The Science and Practice of Pharmacy, The University of the Sciences in Philadelphia, Editor, Lippincott, Williams, & Wilkins, Philadelphia, PA, 21^{st} Edition (2005).

Polypeptide: Any chain of amino acids, regardless of length or post-translational modification (e.g., glycosylation or phosphorylation). With regard to polypeptides and proteins, the word “about” indicates integer amounts. Thus, in one example, a polypeptide “about” 29 amino acids in length is from 28 to 30 amino acids in length. Thus, a polypeptide “about” a specified number of residues can be one amino acid shorter or one amino acid longer than the specified number. A fusion polypeptide includes the amino acid sequence of a first polypeptide and a second different polypeptide (for example, a heterologous polypeptide), and can be synthesized as a single amino acid sequence. A recombinant polypeptide has an amino acid sequence that is not naturally occurring or that is made by two otherwise separated segments of an amino acid sequence.

Recombinant: A nucleic acid or protein that has a sequence that is not naturally occurring or has a sequence that is made by an artificial combination of two otherwise separated segments of sequence (e.g., a “chimeric” sequence). This artificial combination can be accomplished by chemical synthesis or by the artificial manipulation of isolated segments of nucleic acids, for example, by genetic engineering techniques.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are known t. Various programs and alignment algorithms are described in: Smith and Waterman, Adv. Appl. Math. 2:482, 1981; Needleman and Wunsch, 1970, J Mol Biol 48, 443-453; Higgins and Sharp, 1988, Gene 73, 237-244; Higgins and Sharp, 1989, CABIOS 5, 151-153; Corpet et al., 1988, Nucleic Acids Research 16, 10881-10890; and Pearson and Lipman, 1988, Proc Natl Acad Sci USA 85, 2444-2448. Altschul et al., 1994, Nature Genet 6, 119-129, presents a detailed consideration of sequence alignment methods and homology calculations.

The NCBI Basic Local Alignment Search Tool (BLAST) (Altschul et al., 1990, J Mol Biol 215, 403-410) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least 75%, for example at least 80%, sequence identity counted over the full length alignment with the amino acid sequence of a polypeptide using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and can possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, in some examples, variants of a polypeptide or nucleic acid sequence are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid or nucleotide sequence of interest. Sequences with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids (or 30-60 nucleotides), and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Single guide RNA (sgRNA): A synthetic guide RNA used to recognize a target DNA sequence and direct a Cas nuclease to the target. In some examples, the sgRNAs are generated from subcloning an optimized mouse genome-wide lentiviral CRISPR sgRNA library, such as lentiCRISPRv2-Brie (Doench et al., Nat Biotechnol 34:184-191, 2016, herein incorporated by reference in its entirety). In some examples, a sgRNA expressing cassette further comprises a U6 promoter and/or a guide RNA scaffold.

Subject: Living multi-cellular vertebrate organisms, a category that includes human and non-human mammals, such as non-human primates, rats, mice, dogs, cats, horses, cows and pigs. In an example, a subject is a human. In an additional example, a subject is selected that is in need of modulating osteoclast fusion. For example, the subject can need increased or decreased osteoclast fusion, or can need increased or decreased bone resorption.

T cell: A white blood cell (lymphocyte) that is an important mediator of the immune response. T cells include, but are not limited to, cluster of differentiation (CD)4⁺ T cells and CD8⁺ T cells. Mature CD4+ cells, also known as helper T cells, help orchestrate the immune response, including antibody responses as well as killer T cell responses. Mature CD8⁺ T cells can be cytotoxic T cells. Activated T cells can be detected by an increase in cell proliferation and/or expression of or secretion of one or more cytokines (such as IL-2, IL-4, IL-6, IFN-γ, or TNFα). Activation of CD8⁺ T cells can also be detected by an increase in cytolytic activity in response to an antigen.

A “regulatory T (Treg) cell” is a type of T cell that has a role in maintaining immune system homeostasis by suppressing over-reactive immune responses (Josefowicz et al. Annu. Rev. Immunol. 30, 531-564, 2012). Treg can be CD4+CD25+FoxP3+ T cells. Defects in Treg cells lead to autoimmune disorders and immunopathology, whereas certain tumors are enriched with Treg cells that suppress anti-tumor immune responses (Tanaka and Sakaguchi, Cell Res. 27, 109-118, 2017). Treg can also produce cytokines such as transforming growth factor (TGF)-β, interleukin (IL)-35 and IL-10. Other types of Tregs include Tr1 cells, which are CD4+FoxP3−IL10+ TGFβ1+ cells, see Gregori and Roncarolo, Front. Immunol., doi.org/10.3389/fimmu.2018.00233, Feb. 15, 2018, incorporated herein by reference. Vitamin D3 induces differentiation of both Treg and Tr1 cells, see van der Aar et al., J. Allerg. Clin. Immunol. 127(6): 1532-1540.e7, 2011.

### Overview

In some embodiments disclosed is a tolerogenic composition including a1) one or more microparticles; b1) one or more regulatory T cell (Treg) stimulating agents encapsulated within each microparticle; and c1) a CRISPR-Cas effector polypeptide or an immunogenic fragment thereof, or a fusion polypeptide comprising a CRISPR-Cas effector polypeptide or immunogenic fragment.

In some embodiments, the one or more Treg stimulating agents comprise CCL22, retinoic acid, or vasoactive intestinal peptide. In further embodiments, the one more Treg stimulating agents include IL-2, TGF-β, rapamycin, a rapamycin derivative, or a CCR8 ligand. In yet other embodiments, the one or more Treg stimulating factors include TGFβ, IL2, and rapamycin. In further embodiments, the tolerogenic composition includes i) a microparticle comprising TGFβ; ii) a microparticle comprising IL-2, and iii) a microparticle comprising rapamycin.

In some embodiments, the microparticle includes at least one polymer. In further non-limiting examples, at least one polymer comprises polyethylene glycol (PEG), a poly(amino acid), polylactate, polylactic acid, polyglutamic acid, polyglycolic acid (PGA), polylactic acid (PLA), poly(lactic-co-glycolic acid) (PLGA), polyvinyl acetate (PVA), poly(ethylene glycol-b-(DL-lactic acid-co-glycolic acid)-b-ethylene glycol) (PEG-PLGA-PEG), Poly(ethylene glycol)-b-poly(D,L-lactide-co-glycolide) (PEG-b-PLGA), polycaprolactone-PEG (PCL-PEG), poly(vinylidene fluoride)-PEG (PVDF-PEG), poly(lactic acid-co-PEG) (PLA-PEG), poly(methyl methacrylate)-PEG (PMMA-PEG), and combinations thereof. In other embodiments, the microparticle comprises alginate.

In yet other embodiments, the microparticles are approximately 0.5 to 5 μm in diameter and provide sustained release of TGF-β1, rapamycin, and/or IL-2. In particular non-limiting examples, the microparticles provide release for approximately 1 week.

In some embodiments, the CRISPR-Cas effector polypeptide is a type II CRISPR-Cas effector polypeptide, a type V CRISPR-Cas effector polypeptide, or a type VI CRISPR-Cas effector polypeptide. In other embodiments, the CRISPR-Cas effector fusion polypeptide incudes: i) a CRISPR-Cas effector polypeptide; and ii) one or more heterologous effector polypeptides. In some embodiments, at least one of the one or more heterologous effector polypeptides is a single stranded nuclease, a double strand nuclease, a helicase, a methylase, a demethylase, an acetylase, a deacetylase, a deaminase, an integrase, a recombinase, a base editor, or a prime editor.

In further embodiments, methods are disclosed inducing tolerance to a CRISPR-Cas effector polypeptide in a mammalian subject, the method comprising administering to the subject an effective amount of a tolerogenic composition as disclosed herein, thereby inducing tolerance to the CRISPR-Cas effector polypeptide. In some embodiments, the composition is administered intradermally, subdermally, subcutaneously, or intramuscularly.

In other embodiments, the tolerogenic composition includes a2) a dissolvable microneedle array; b2) one or more agents that promote differentiation of tolerogenic DCs; or c2) a CRISPR-Cas effector polypeptide or immunogenic fragment thereof, or a fusion polypeptide comprising a CRISPR-Cas effector polypeptide.

In some embodiments, the dissolvable microneedle array includes: i) a substrate comprising a biocompatible material that forms base portion; and ii) a plurality of microneedles extending from the base portion. In further embodiments, the biocompatible material includes carboxymethylcellulose, polyvinylpyrrolidone (PVP), polyvinyl alcohol (PVA), hyaluronic acid (HA), or gelatin. In a specific non-limiting example, the biocompatible material includes carboxymethylcellulose.

In more embodiments, the one or more agents that promote differentiation of tolerogenic dendritic cells include vitamin or an analog thereof, such as vitamin D3 or a vitamin D3 analog.

In some embodiments, methods are disclosed for inducing tolerance to a CRISPR-Cas effector polypeptide in a mammalian subject, including administering to the subject an effective amount of the tolerogenic composition that includes the microneedle array. In more embodiments, the composition is administered locally, such as intradermally or intracutaneously.

In yet other embodiments, the tolerogenic composition is administered repeatedly to the subject. In non-limiting examples, the tolerogenic composition is administered in a prime-boost strategy to the subject.

In further embodiments, the disclosed methods include administering to the subject a gene-editing composition comprising the CRISPR-Cas effector polypeptide following administering the effective amount of the tolerogenic composition, wherein the tolerogenic composition induces tolerance to the CRISPR-Cas effector polypeptide present in the gene-editing composition. In some embodiments, the gene-editing composition is administered to the subject within about 6 months of administration of the tolerogenic composition. The subject can be a human. The subject can be a non-human mammal.

### CRISPR-Cas Effector Polypeptides

The disclosed methods use a tolerogenic composition including one or more CRISPR-Cas effector polypeptides or immunogenic fragments thereof, or one or more fusion polypeptides comprising a CRISPR-Cas effector polypeptide. A CRISPR-Cas effector polypeptide suitable for use is a class 2 CRISPR effector polypeptide, also referred to herein as a class 2 CRISPR-Cas effector polypeptide. For example, in some cases, the CRISPR-Cas effector polypeptide is a type II CRISPR-Cas effector polypeptide. In some cases, the type II CRISPR-Cas effector polypeptide is a Cas9 polypeptide. In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide. a Cas13b polypeptide, a Cas13c polypeptide, or a Cas13d polypeptide. In some cases, the CRISPR-Cas effector polypeptide is a Cas14 polypeptide. In some cases, the CRISPR-Cas effector polypeptide is a Cas14a polypeptide, a Cas14b polypeptide, or a Cas14c polypeptide. A CRISPR-Cas effector polypeptide suitable for use includes a CRISPRi polypeptide. See, e.g., Qi et al. (2013) Cell 152:1173; and Jensen et al. (2021) Genome Research doi:10.1101/gr.275607.121. A CRISPR-Cas effector polypeptide suitable for use includes a CRISPRa polypeptide. See, e.g., Jensen et al. (2021) Genome Research doi:10.1101/gr.275607.121; and Breinig et al. (2019) Nature Methods 16:51. A CRISPR-Cas effector polypeptide suitable for use includes a CRISPRoff polypeptide. See, e.g., Nuñez et al. (2021) Cell 184:2503. A CRISPR-Cas effector polypeptide suitable for use includes a nickase. A CRISPR-Cas effector polypeptide suitable for use includes a catalytically inactive CRISPR-Cas effector polypeptide that retains binding (when complexed with a guide RNA) to a target nucleic acid. A CRISPR-Cas effector polypeptide suitable for use includes a fusion polypeptide comprising: i) a CRISPR-Cas effector polypeptide; and ii) one or more heterologous fusion partners (also referred to as “heterologous polypeptides”). Non-limiting examples of Cas nucleases include Cas1, Cas1B, Cas2, Cas3, Cas4, Cas5, Cas6, Cas7, Cas8, Cas9 (also known as Csn1 and Csx12), Cas10, Cas13d, Cpf1, C2c3, C2c2 and C2c1Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, Cas lambda, Cas phi, Cas X, and Cas Y, as well as homologs thereof. Immunogenic fragments of these CRISPR-Cas effector polypeptides can also be included in the disclosed compositions.

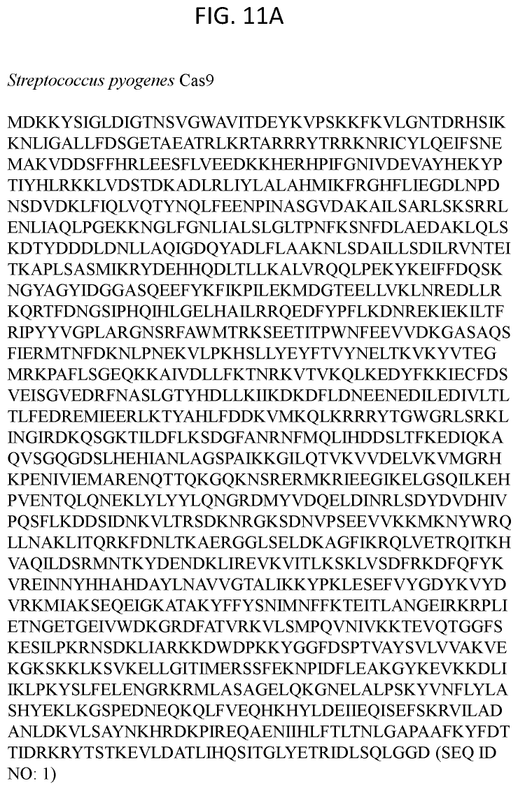

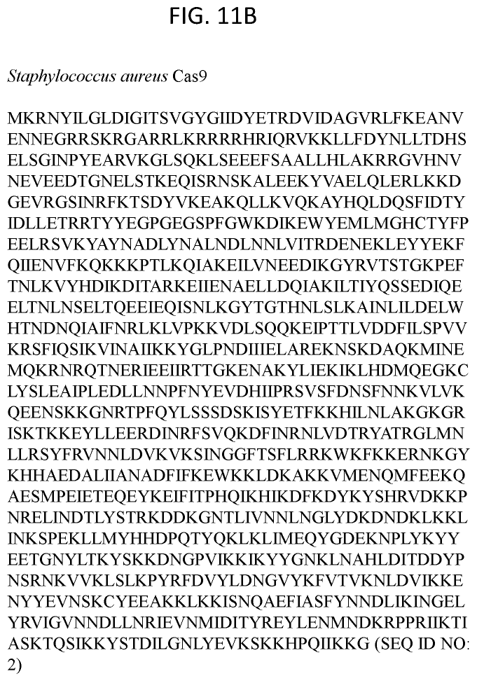

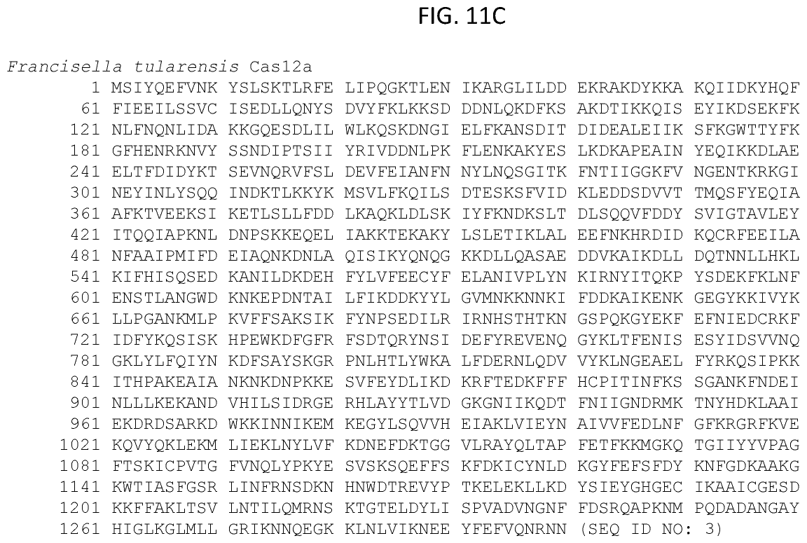

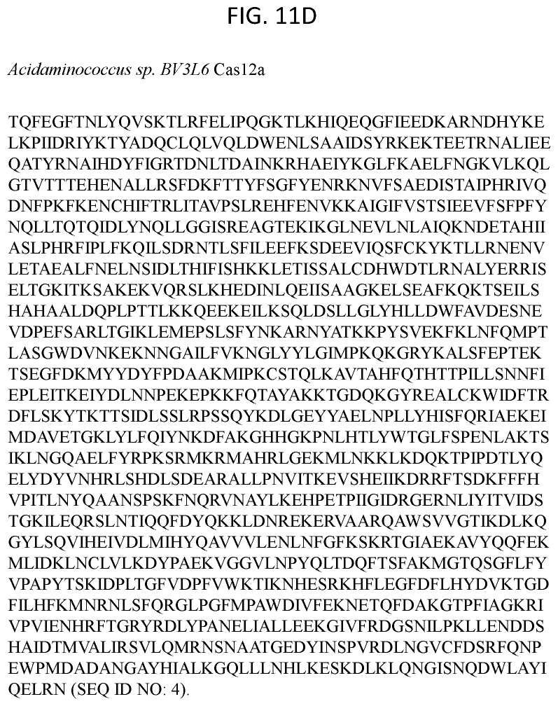

In some cases, a CRISPR-Cas effector polypeptide suitable for inclusion in a composition of the present disclosure is a Cas9 polypeptide. In some cases, a Cas9 polypeptide comprises an amino acid sequence having at least 50%, at least 60%, at least 70%, at least 80%, at least 90%, at least 95%, at least 98%, at least 99%, or more than 99%, amino acid sequence identity to the Streptococcus pyogenes Cas9 amino acid sequence (SEQ ID NO: 1) depicted in FIG. 11A.

In some cases, the Cas9 polypeptide is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%. amino acid sequence identity to any known saCas9 amino acid sequence, e.g., a saCas9 amino acid sequence (SEQ ID NO: 2) depicted in FIG. 11B.

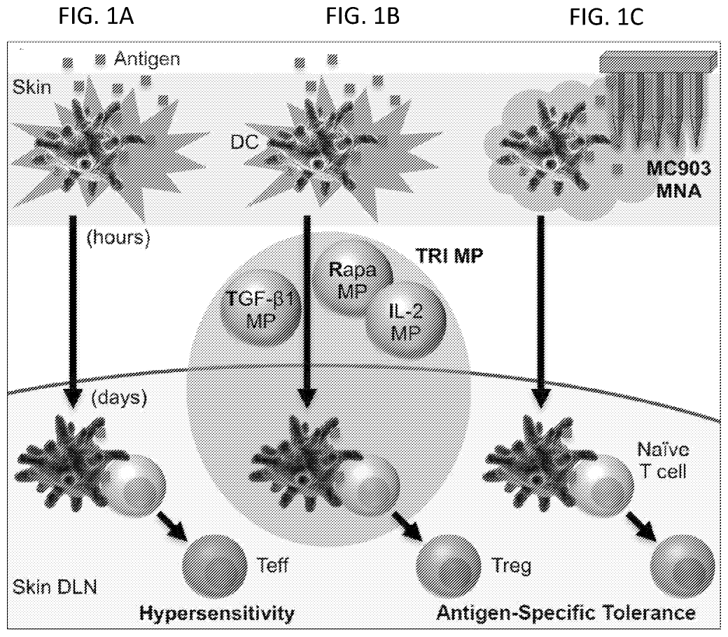

In some cases, a suitable Cas9 polypeptide is a high-fidelity (HF) Cas9 polypeptide. Kleinstiver et al. (2016) Nature 529:490. For example, amino acids N497, R661, Q695, and Q926 of the amino acid sequence depicted in FIG. 1A are substituted, e.g., with alanine. For example, an HF Cas9 polypeptide can comprise an amino acid sequence having at least 90%, at least 95%, at least 98%, or at least 99%, amino acid sequence identity to the amino acid sequence depicted in FIG. 1A, where amino acids N497, R661, Q695, and Q926 are substituted, e.g., with alanine. In some cases, a suitable Cas9 polypeptide exhibits altered PAM specificity. See, e.g., Kleinstiver et al. (2015) Nature 523:481.

In some cases, a suitable Cas9 polypeptide comprises an R691A substitution. For example, in some cases, a suitable Cas9 polypeptide comprises an amino acid sequence having at least 90%, at least 95%, at least 98%, or at least 99%, amino acid sequence identity to the amino acid sequence depicted in FIG. 11A, where amino acid 691 is Ala.

In some cases, a suitable Cas9 polypeptide comprises D1135V, R1335Q, and T1337R substitutions. For example, in some cases, a suitable Cas9 polypeptide comprise an amino acid sequence having at least 90%, at least 95%, at least 98%, or at least 99%, amino acid sequence identity to the amino acid sequence depicted in FIG. 1A, where amino acid 1135 is Val, amino acid 1335 is Gln, and amino acid 1337 is Arg, and where the Cas9 polypeptide exhibits relaxed PAM requirements.

In some cases, a suitable Cas9 polypeptide is a SpRY variant. See, e.g., Zhang and Zhang (2020) Trends Genetics 36:546; and Walton et al. (2020) Science 368:290; and U.S. Patent Publication No. 2021/0284978. SpRY is a variant of S. pyogenes Cas9; this variant has reduced PAM requirements. For example, in some cases, a suitable Cas9 polypeptide comprises D1135L, S1136W, G1218K, E1219Q, R1335Q, and T1337R substitutions. For example, in some cases, a suitable Cas9 polypeptide comprise an amino acid sequence having at least 90%, at least 95%, at least 98%, or at least 99%, amino acid sequence identity to the amino acid sequence depicted in FIG. 11A, where amino acid 1135 is Leu, amino acid 1136 is Trp, amino acid 1218 is Lys, amino acid 1219 is Gln, amino acid 1335 is Gln, and amino acid 1337 is Arg. In some cases, a suitable Cas9 polypeptide comprise an amino acid sequence having at least 90%, at least 95%, at least 98%, or at least 99%, amino acid sequence identity to the amino acid sequence depicted in FIG. 11A; and comprises amino acid substitution(s) at one, two, three, four, five, or all six of the following positions: at E1219 (e.g., an E1219Q, an E1219H, an E1219S, or an E1219V substitution); S1136 (e.g., an S1136W, an S1136F, an S1136A, or an S1136V substitution); D1135 (e.g., a D1135L, a D1135A, a D1135W, or a D1135F substitution): G1218 (e.g., a G1218R, a G1218K, or a G1218S substitution); R1335 (e.g., an R1335Q substitution); and T1337 (e.g., a T1337R or a T1337K substitution).

In some cases, a suitable Cas9 polypeptide is a Cas9 polypeptide or a Cas9-NG polypeptide. See, e.g., Zhong et al. (2019) Molec. Plant 12:1027; Hu et al. (2018) Nature 556:57; and Nishimasu et al. (2018) Science 361:1259. Cas9 nucleic acid and protein sequences are publicly available. For example, GENBANK® Accession Nos. nucleotides 796693 . . . 800799 of CP012045.1 and nucleotides 1100046 . . . 1104152 of CP014139.1 disclose Cas9 nucleic acids, and GENBANK® Accession Nos. AMA70685.1 and AKP81606.1 disclose Cas9 proteins. In some examples, the Cas9 is a deactivated form of Cas9 (dCas9), such as one that is nuclease deficient (e.g., those shown in GENBANK® Accession Nos. AKA60242.1 and KR011748.1). In certain examples, Cas9 has at least 80% sequence identity, for example at least 85%, at least 90%, at least 95%, at least 96%, at least 97%, at least 98%, or at least 99% sequence identity to such sequences, and retains the ability to cut DNA.

In some cases, a suitable CRISPR-Cas effector polypeptide is a type V CRISPR-Cas effector polypeptide. In some cases, a type V CRISPR-Cas effector polypeptide is a Cas 12a protein. In some cases, a Cas12a protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to any known Cas12a protein, e.g., a Cas12a amino acid sequence depicted in FIG. 11C or FIG. 11D.

In some cases, the CRISPR-Cas effector polypeptide is a CRISPR-Cas effector fusion polypeptide comprising: a) a CRISPR-Cas effector polypeptide or immunogenic fragment thereof; and b) one or more heterologous polypeptides (also referred to as fusion partners). In some cases, the one or more heterologous polypeptides comprises a single stranded nuclease, a double strand nuclease, a helicase, a methylase, a demethylase, an acetylase, a deacetylase, a deaminase, an integrase, a recombinase, a base editor, or a prime editor. In some cases, the one or more heterologous polypeptides comprises a nuclear localization signal. In some cases, the fusion partner (heterologous polypeptide) is a reverse transcriptase. In some cases, the fusion partner is a base editor. In some cases, the fusion partner (heterologous polypeptide) is a deaminase. The fusion polypeptide can also include a suitable carrier.

CRISPR-Cas effector polypeptides are known in the art that have reduced immunogenicity as compared to a parental CRISPR-Cas effector polypeptide, see PCT Publication No. WO 2017/081288A1, incorporated by reference. These recombinant CRISPR-Cas effector polypeptides include one or more amino acid substitutions in one or more residues corresponding to one or more MHC Class I and/or MHC Class Il binding sites in a wild-type CRISPR-Cas effector polypeptide, wherein the recombinant CRISPR-Cas effector polypeptide has reduced immunogenicity compared to a wild-type CRISPR-Cas effector polypeptide.

Immunogenic fragments of a CRISPR-Cas effector polypeptide are also of use, such as an immunogenic fragment that corresponds to the MHC Class I and/or Class II binding site. The immunogenic fragment can include 9 (9-mer) or 10 (10-mer) amino acids. The immunogenic fragment can induce a tolerogenic immune response. In further embodiments, the fragment is 8, 9, 10 11 or 12 amino acids in length. Mixtures of immunogenic fragments are also of use. The mixture can include at least 2, 3, 4, 5, 6, 7, 8, 9 or 10 different fragments. These immunogenic fragments can be used to induce a tolerogenic immune response.

For treatment of a subject, depending on activity of the one or more CRISPR-Cas effector polypeptides or immunogenic fragments thereof, or one or more fusion polypeptides, the manner of administration, age and body weight of the patient, different doses may be necessary. Under certain circumstances, higher or lower doses may be appropriate. The administration of the dose can be carried out both by single administration in the form of an individual dose unit or else several smaller dose units and also by multiple administrations of subdivided doses at specific intervals. A skilled clinician can readily determine an effective dose to induce tolerance.

For administration to a subject, an effective amount of one or more CRISPR-Cas effector polypeptides or immunogenic fragments thereof, or one or more fusion polypeptides comprising a CRISPR-Cas effector polypeptide or immunogenic fragment thereof can be included in a pharmaceutically acceptable carrier. These pharmaceutical compositions can be prepared and administered in dose units. Liquid formulations are generally of use. Solid dose units are tablets, capsules, single injectables and even suppositories. A suitable administration format may best be determined by a medical practitioner for each subject individually. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42: 2S, 1988. The dosage form of the pharmaceutical composition will be determined by the mode of administration chosen. Generally, the pharmaceutical compositions include an effective amount of the CRISPR-Cas effector polypeptides, immunogenic fragments, and fusion polypeptides.

Suitable solid or liquid pharmaceutical preparation forms are, for example, granules, powders, tablets, coated tablets, (micro)capsules, suppositories, syrups, emulsions, suspensions, creams, aerosols, drops or injectable solution in ampoule form and also preparations with protracted release of one or more CRISPR-Cas effector polypeptides or immunogenic fragments thereof, or one or more fusion polypeptides comprising a CRISPR-Cas effector polypeptide, in whose preparation excipients and additives and/or auxiliaries such as disintegrants, binders, coating agents, swelling agents, lubricants, flavorings, solubilizers or scaffolds are customarily used. The pharmaceutical compositions are suitable for use in a variety of drug delivery systems. For a brief review of present methods for drug delivery, see Langer, Science 249:1527-1533, 1990.

The present disclosure is not limited to CRISPR-Cas effector polypeptides, immunogenic fragments, and fusion polypeptides. The present disclosed compositions and methods are of use with other systems, such as zinc-finger nucleases (ZFNs), and transcription activator-like effector nucleases (TALENs), amongst others.

### Microparticles

Tolerogenic compositions are provided that include one or more microparticles, one or more Treg stimulating agents encapsulated within the one or more microparticles, and a CRISPR-Cas effector polypeptide or immunogenic fragment thereof, or a fusion polypeptide comprising a CRISPR-Cas effector polypeptide or immunogenic fragment thereof. Illustrative Treg stimulating agents include the C-C chemokine motif (CCL22), interleukin 2 (IL-2), rapamycin, transforming growth factor beta (TGF-β), retinoic acid, and vasoactive intestinal peptide (VIP).

In certain embodiments, Treg stimulating agents are highly effective at recruiting Tregs. In particular, CCL22, retinoic acid and VIP recruit Tregs. In certain embodiments, the therapeutic agent-loaded microparticles are highly effective at recruiting Tregs. The microparticles are effective at recruiting endogenous Treg cells to a local site.

In certain embodiments, the Treg stimulating agent is highly effective at inducing Tregs. In particular, IL-2, rapamycin and TGF-β are effective at inducing Tregs. In certain embodiments, the therapeutic agent-loaded microparticles are highly effective at stimulating Tregs. The dissolvable microspheres are effective at stimulating endogenous Treg cells at a local site.

In certain embodiments, without being bound by theory, tolerance is induced by the induction of a subject's own Tregs from naïve CD4⁺ T cells. This approach utilizes the body's own natural mechanism to differentiate peripheral naïve CD4⁺ T cells into Tregs through a subset of antigen presenting cells known as tolerogenic dendritic cells (tDCs). Specifically, tDCs can induce differentiation of Tregs through the secretion of IL-2 and TGF-β cytokines. However, the maintenance of Tregs is somewhat more complex and depends on a local microenvironment that is not only favorable to differentiation of Tregs, but also unfavorable to differentiation into other effector T cells. One-way to ensure that cells do not differentiate into pathogenic effector T cells is through the small molecule, rapamycin. Rapamycin (Rapa) is an mTOR inhibitor that can suppress the generation and proliferation of effector T cells. Rapamycin derivatives are also of use and are known in the art.

In certain embodiments, the body's own endogenous Tregs are enriched by delivering a combination of Treg inducing agents through TRI microspheres (TGF-β1, Rapamycin (Rapa) or a derivative thereof, and IL-2), which are microparticles encapsulating TGF-β1, Rapa and IL-2. In some embodiments, TGF-β1, Rapa and IL-2 are included in separate microparticles, and a mixture is administered. Thus, in some embodiments, the tolerogenic composition comprises i) a microparticle comprising TGFβ; ii) a microparticle comprising IL-2, and iii) a microparticle comprising rapamycin (or a derivative thereof). Without being bound by theory, this system is able to increase the prevalence of Tregs and, in turn, induce tolerance.

One or more microparticles can be delivered via subcutaneous or intramuscular injection. The microparticles can be delivered to a mucosal surface, such as in a gel. The one or more microparticles can be delivered by local injection or a local retention system. The one or more microparticles are administered at/near a site of in vivo gene editing. The one or more microparticles can be deleted at the same location/site as the CRISPR-Cas effector polypeptide, immunogenic fragment thereof, or a fusion polypeptide.

In certain embodiments, the amount of one or more Treg stimulating agents loaded into the one or more microparticles may range from about 1 ng to about 1 mg, more particularly about 1 ng to about 100 μg agent per mg of microparticles. In certain specific embodiments, the amount of one or more Treg stimulating agents loaded into the one or more microparticles is about 25 ng-7.5 μg agent per mg of microparticles. The Treg stimulating agent can be IL-2 or TGF-β1. The Treg stimulating agents can be included in separate microparticles or included in the one microparticle, including multiple Treg stimulating agents. As used herein, administration of “a microparticle” is directed to the administration of one or more microparticles including the Treg stimulating agents.

Suitable doses include, but are not limited to, about 5 μg to about 100 μg of IL-2 per about 200 mg polymer. Suitable doses include, but are not limited to, about 5, 10, 15, 20, 25, 30, 35, 40, 45, 50, 55, 60, 65, 70, 75, 80, 95, 100, 110, 120, 130, 140, 150, 160, 170, 180, 190, or 200 μg of IL-2 per about 200 mg of polymer. Suitable doses include, but are not limited to, about 5 μg to about 100 μg of TGF-β1 per about 200 mg polymer. Suitable doses include, but are not limited to, about 5, 10, 15, 20, 25, 30, 35, 40, 45, 50, 55, 60, 65, 70, 75, 80, 95, 100, 110, 120, 130, 140, 150, 160, 170, 180, 190, or 200 μg of TGF-β1 per about 200 mg of polymer. Suitable doses include, but are not limited to, about 1.5 mg to about 2 mg rapamycin per about 200 mg of polymer. Suitable doses also include, but are not limited to, about 7.5 μg to about 2 mg rapamycin per about 200 mg of polymer. Suitable doses also include, but are not limited to, about 10 μg rapamycin per mg of polymer.

In yet other embodiments, the microparticles are approximately 0.5 to 5 μm in diameter and provide sustained release of TGF-β1, rapamycin, and/or IL-2. In particular examples, the microparticles provide release for approximately 1 week.

The microparticles include at least one polymer. The polymers for the microparticle may be bioerodible polymers so long as they are biocompatible. Bio-erodible polymers include polyhydroxyacids such as polylactic acid and copolymers thereof. Illustrative polymers include poly glycolide, poly lactic acid (PLA), and poly (lactic-co-glycolic acid) (PLGA). Another class of approved biodegradable polymers is the polyhydroxyalkanoates. Other suitable polymers include, but are not limited to: polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses. polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate) poly(lauryl methacrylate), poly(phenyl methacrylate), poly(methyl acrylate), poly(isopropyl acrylate), poly(isobutyl acrylate), poly(octadecyl acrylate), polyethylene, polypropylene polyethylene glycol), poly(ethylene oxide), poly(ethylene terephthalate), poly(vinyl alcohols), poly(vinyl acetate), poly vinyl chloride polystyrene, polyvinylpryrrolidone, alginate, poly(caprolactone), dextran and chitosan. In some non-limiting examples, the microparticles can include alginate. In other non-limiting examples, the polymer is an ester-terminated PLGA.

In more embodiments, the polymer is a polyethylene glycol-poly(lactic-co-glycolic acid) copolymer. In further embodiments, the at least one polymer comprises polyethylene glycol (PEG), a poly(amino acid), polylactate, polylactic acid, polyglutamic acid, polyglycolic acid (PGA), polylactic acid (PLA), poly(lactic-co-glycolic acid) (PLGA), polyvinyl acetate (PVA), poly(ethylene glycol-b-(DL-lactic acid-co-glycolic acid)-b-ethylene glycol) (PEG-PLGA-PEG), Poly (ethylene glycol)-b-poly(D,L-lactide-co-glycolide) (PEG-b-PLGA), polycaprolactone-PEG (PCL-PEG), poly(vinylidene fluoride)-PEG (PVDF-PEG), poly(lactic acid-co-PEG) (PLA-PEG), poly(methyl methacrylate)-PEG (PMMA-PEG), and combinations thereof.

The percent loading may be increased by “matching” the hydrophilicity or hydrophobicity of the polymer to the agent to be encapsulated. In some cases, such as PLGA, this can be achieved by selecting the monomer ratios so that the copolymer is more hydrophilic for hydrophilic drugs or less hydrophilic for hydrophobic drugs. Alternatively, the polymer can be made more hydrophilic, for example, by introducing carboxyl groups onto the polymer. In non-limiting examples, the combination of a hydrophilic drug and a hydrophobic drug can be encapsulated in microparticles prepared from a blend of a more hydrophilic PLGA and a hydrophobic polymer, such as PLA.

In some embodiments, the polymer is a PLGA copolymer or a blend of PLGA and PLA. The molecular weight of PLGA is from about 10 kD to about 80 kD, more preferably from about 10 kD to about 35 kD. The molecular weight range of PLA is from about 20 to about 30 kDa. The ratio of lactide to glycolide is from about 75:25 to about 50:50. In one embodiment, the ratio is 50:50. Illustrative polymers include, but are not limited to, poly(D,L-lactic-co-glycolic acid) (PLGA, 50:50 lactic acid to glycolic acid ratio, Mₙ=10 kDa, acid-terminated, referred to as 502H); poly(D,L-lactic-co-glycolic acid) (PLGA, 50:50 lactic acid to glycolic acid ratio, Mₙ=25 kDa, acid-terminated, referred to as 503H); poly(D,L-lactic-co-glycolic acid) (PLGA, 50:50 lactic acid to glycolic acid ratio, Mₙ=30 kDa, acid-terminated, referred to as 504H); poly(D,L-lactic-co-glycolic acid) (PLGA, 50:50 lactic acid to glycolic acid ratio, Mₙ=35 kDa, ester-terminated, referred to as 504); and poly(D,L-lactic-co-glycolic acid) (PLGA, 75:25 lactic acid to glycolic acid ratio, Mₙ=10 kDa, referred to as 752).

A microparticle can range in diameter between about 0.1 μm and about 1000 μm or any range therebetween, such as between 0.1 μm and 0.5 μm, 0.5 μm and 1.0 μm, about 1.0 μm and about 5.0 μm, about 5.0 μm and about 10.0 μm, about 10.0 μm and about 20.0 μm, about 20.0 μm and about 35.0 μm, about 35.0 μm and about 50.0 μm, about 50.0 μm and about 75.0 μm, about 75.0 μm and about 100.0 μm, about 100.0 μm and about 250.0 μm, about 250.0 μm and about 500.0 μm, about 500.0 μm and about 750 um, or about 750.0 μm and about 1000.0 μm. In one embodiment, the microparticle ranges in diameter between about 0.1 μm and 1 μm such as between about 0.2 μm and about 1 μm, between about 0.3 μm and about 1 μm, between about 0.4 μm and about 1 μm, or between about 0.5 μm and about 1 μm. In another embodiment, the microparticle ranges in diameter between about 0.1 μm and 5 μm such as between about 0.2 μm and about 5 μm, between about 0.3 μm and about 5 μm, between about 0.4 μm and about 5 μm, or between about 0.5 μm and about 5 μm. In certain embodiments, loaded microparticles may have a volume average diameter of 200 nm to 30 μm, more particularly 1 to 10 μm. In certain embodiments, the agent-loaded microparticles do not have a volume average diameter of 10 μm or greater. The microparticles can be, for example, about 15 μm to 30 μm in diameter, such as about 15, 20, 25, oe 30 μm in diameter. The microparticles can be, for example, about 0.5 μm to 5 μm in diameter, such as about 0.5, 1, 2, 3, 4, or 5 μm in diameter. The microparticles can be, for example, about 2 μm in diameter. The agent-loaded microparticles may be pore less or they may contain varying amounts of pores of varying sizes, typically controlled by adding NaCl during the synthesis process.

The agent-loaded microparticle fabrication method can be single or double emulsion depending on the desired encapsulated agent solubility in water, molecular weight of polymer chains used to make the microparticles (MW can range from ˜1000 Da to over 100,000 Da) which controls the degradation rate of the microparticles and subsequent drug release kinetics.

Microparticles of use in the method disclosed herein may provide for sustained release. For example, the sustained release may be over a period of at least one day, more particularly at least 5 days or at least 10 days, and most particularly at least 30 days. The agent release can be linear or non-linear (single or multiple burst release). In certain embodiments, the agent may be released without a burst effect. For example, the sustained release may exhibit a substantially linear rate of release of the therapeutic agent in vivo over a period of at least one day, more particularly at least 5 days or at least 10 days, and most particularly at least 30 days. By substantially linear rate of release it is meant that the therapeutic agent is released at a rate that does not vary by more than about 20% over the desired period of time, more usually by not more than about 10%. It may be desirable to provide a relatively constant rate of release of the agent from the delivery system over the life of the system. For example, it may be desirable for the agent to be released in amounts from 0.1 to 100 μg per day, more particularly 1 to 10 μg per day, for the life of the system. However, the release rate may change to either increase or decrease depending on the formulation of the polymer microparticle

In certain embodiments, the delivery system may release an amount of an agent that is effective in providing a local concentration in a range from 1 μg/ml to 200 μg/ml, such as 1 to 5 μg/ml. Specific, non-limiting embodiments are about 10 ng/ml IL-2, about 5 ng/ml of TGF-β1, and about 10 ng/ml rapamycin (see Jhunjhunwala et al., J. Controlled Release 159(1): 78-84. 2012). In certain embodiments, there is no initial lag phase of release. The desired release rate and target drug concentration can vary depending on the particular agent chosen.

The microparticles of use in the methods disclosed herein can provide for sustained release of an agent. The term “sustained release” or “controlled release” as used herein, refers to the escape of any attached or encapsulated agent at a predetermined rate. For example, a sustained release of an agent may occur resulting from the predicable biodegradation of a polymer particle (i.e., for example, an artificial antigen presenting cell). The rate of biodegradation may be predetermined by altering the polymer composition and/or ratios comprising the particle. Consequently, the sustained release may be short term or the controlled release may be long term.

In one embodiment, the short term release is between 30 minutes-1 hour. In one embodiment, the short term release is between 1 hour-3 hours. In one embodiment, the short term release is between 3 hours-10 hours. In one embodiment, the short term release is between 10 hours-24 hours.

In one embodiment, the long term release is between 24 hours-36 hours. In one embodiment, the long term release is between 3 days-7 days. In one embodiment, the long term release is between 7 days-1 month. In some embodiments, the microparticles provide sustained release of one or more Treg stimulating agents, such as for at least about 1 day, 2 days, 3 days, 4 days, 5 days, 6 days, 1 week, 2 weeks, 3 weeks, 4 weeks, 1 month, 2 months or 3 months. In some embodiments, the long term release is about 1 week, such as about 2 days to about one week, about 3 days to about one week, about 4 days to about one week, about 5 days to about one week, or about six days to about one week. The long term release can be for less than about a month.

In one embodiment, the long term release is between 1 month-6 months. In one embodiment, the long term release is between 6 months-1 year. In one embodiment, the long term release is at least one month.

The tolerogenic composition including microparticles, as disclosed herein, may include an excipient component, such as effective amounts of buffering agents, and antioxidants for protection from the effects of ionizing radiation during sterilization. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents are advantageously present in amounts sufficient to maintain a pH of the system of between about 2 to about 9 and more preferably about 4 to about 8. As such the buffering agent may be as much as about 5% by weight of the total system. Suitable water-soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from 0.001 to about 5% by weight and preferably 0.01 to about 2% by weight.

In certain embodiments, the microparticles disclosed herein may be administered via injection. Injection sites include but are not limited to intradermal, subdermal, subcutaneous, or intramuscular administration. In some embodiments, the tolerogenic composition comprising a microparticle is delivered via intravenous administration.

### Microneedle Arrays

A CRISPR-Cas effector polypeptide, immunogenic fragment thereof, or a fusion polypeptide, can be administered in dissolvable microneedle array. See, for example, U.S. Published Patent Application No. US-2016-0271381-A1, which is incorporated herein by reference, and PCT Publication No. WO2020/232394, incorporated herein by reference. In some embodiments, the microneedle array is a tip-loaded microarray, which can be prepared using micromilled master molds and spin-molds, see U.S. Published Patent Application No. US-2016-0271381-A1. As disclosed in PCT Publication No. WO 2020/232394, an undercut, or anchor feature, can improve skin retention during application and can be achieved without also interfering with the processing steps, thereby allowing direct removal of the MNAs from the molds. Examples of the utility of microneedle devices include, for example, (1) simultaneous delivery of the disclosed CRISPR-Cas effector polypeptide or immunogenic fragment thereof, or a fusion polypeptide comprising a CRISPR-Cas effector polypeptide or immunogenic fragment thereof, and optionally other agents to generate a tolerogenic immune response and (2) localized skin delivery.

Dissolvable microneedle arrays enable efficient and safe delivery to the skin and mucosal surfaces. A fully-dissolvable microneedle array substrate and unique microneedle geometries can be utilized that enable effective delivery of the CRISPR-Cas effector polypeptide or immunogenic fragment thereof, or a fusion polypeptide comprising a CRISPR-Cas effector polypeptide or immunogenic fragment thereof. This technology can also uniquely enable the simultaneous co-delivery of multiple chemically distinct agents for polyfunctional drug delivery. The agent can promote the differentiation of tolerogenic dendritic cells, such as by inducing hypo-responsiveness to subsequence antigen exposure, or inducing T cells to differentiate into Tregs.

In some embodiments, the agent is vitamin D or an analog thereof, such as vitamin D3, or a vitamin D3 analog. Vitamin D3 has the following structure:

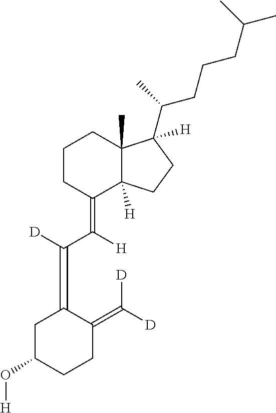 [SMILES: [2H]C([2H])=C1CC[C@H](O)C/C1=C([2H])/C=C1\CCC[C@]2(C)[C@@H]([C@H](C)CCCC(C)C)CC[C@@H]12]

Calcipotriene (also known as calcipotriol or MC-903) is a vitamin D3 analog that is known in the art. Calcipotriene has the following structure:

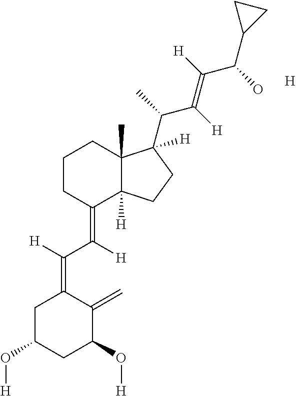

See. e.g., Masuda et al. (1994) J. Biol. Chem. 269:479.

Alfacalcidol can also be utilized. Alfacalcidol has the following structure:

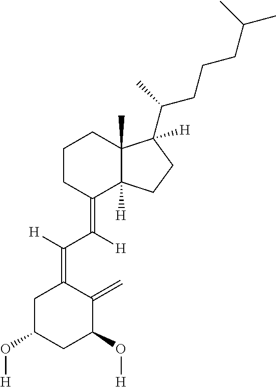 [SMILES: C=C1/C(=C\C=C2/CCC[C@]3(C)[C@@H]([C@H](C)CCCC(C)C)CC[C@@H]23)C[C@@H](O)C[C@@H]1O]

Tacalcitol can also be utilized. Tacalcitol has the following structure:

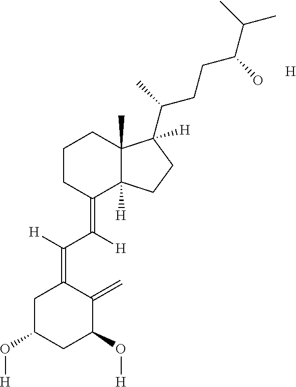

Other suitable agents that promote differentiation of tolerogenic DCs include, but are not limited to, retinoic acid, dexamethasone, IL-10, and TGF-β.

In some embodiments, provided herein is a dissolvable microneedle array for transdermal insertion, e.g., local cutaneous delivery, into a subject for promoting tolerance to Cas9 in a subject in need thereof. The array includes a base portion and a plurality of microneedles extending from the base portion and containing a CRISPR-Cas effector polypeptide, immunogenic fragment thereof, or a fusion polypeptide, and optionally an additional agent.

Microneedles can be pre-formed to have a shape that comprises a first cross-sectional dimension at a top portion, a second cross-sectional dimension at a bottom portion, and a third cross-sectional dimension at an intermediate portion, wherein the intermediate portion is located between the top portion and the bottom portion, and the third cross-sectional dimension is greater than the first and second cross-sectional dimensions.

In yet other embodiments, each microneedle comprises a plurality of layers of dissoluble biocompatible material. In some embodiments, a fabrication technology is utilized that results in various active components to be incorporated into the needle tips, see U.S. Published Patent Application No. US-2016-0271381-A1, which is incorporated herein by reference. Thus, by localizing the active components in this manner, the remainder of the microneedle array volume includes less expensive matrix material that is non-active and generally regarded as safe. The net result is greatly improved efficiency of drug delivery based on (1) reduced waste of non-deliverable active components incorporated into the non-needle portions of the microneedle array, and (2) higher drug concentration in the skin penetrating needle tips.

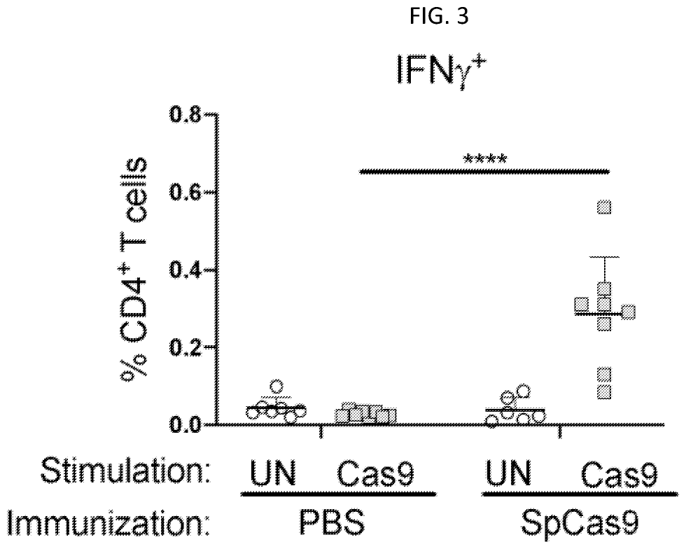

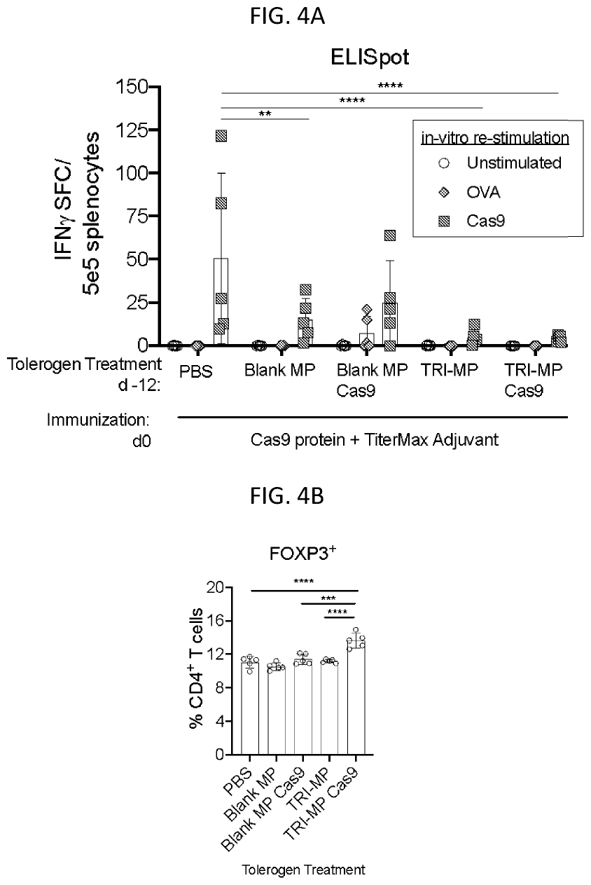

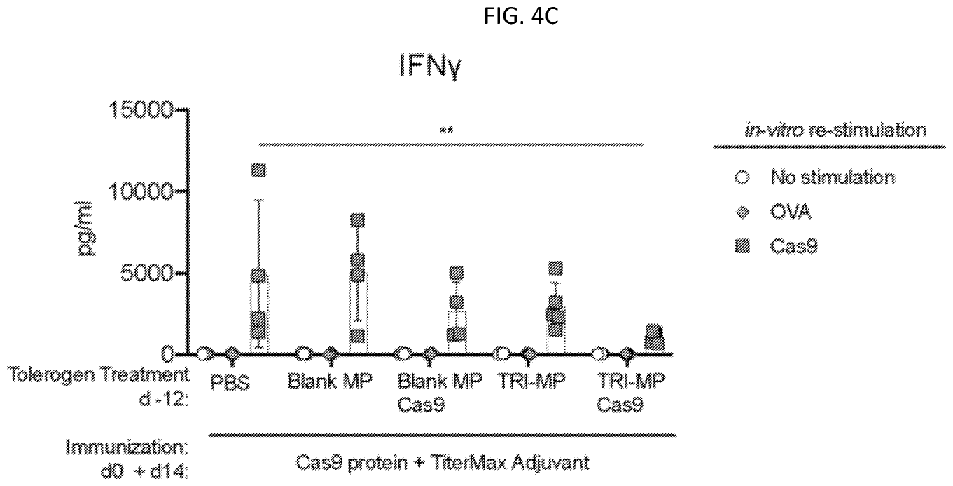

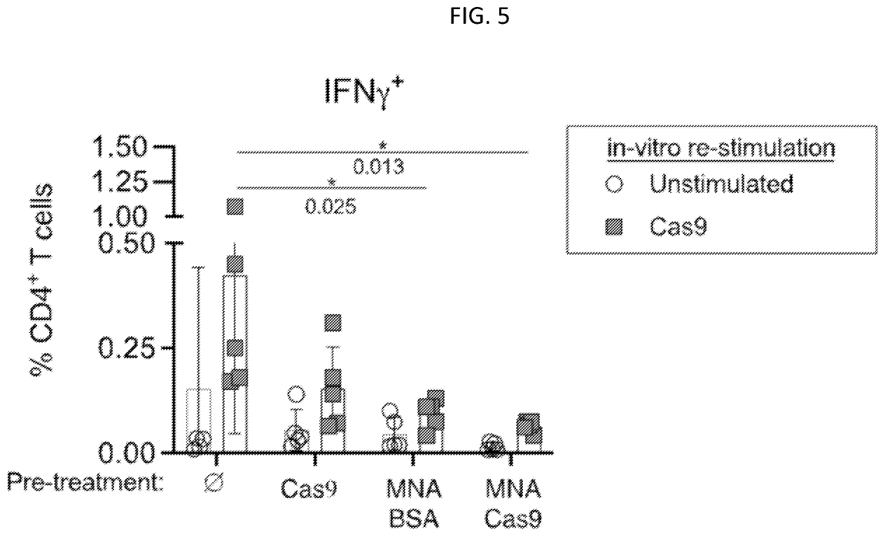

Thus, in some embodiments, the active component is concentrated in the microneedle tips of the respective arrays. Thus, in contrast to conventional microneedle arrays, the active component is not present at even concentration throughout the microneedle array since there is little or no active component present in the supporting base structure. In addition, in some embodiments (as shown, for example, in FIGS. 3A, 3B, 4A, and 4B of U.S. Published Patent Application No. US-2016-0271381-A1, which is incorporated herein by reference), not only is there little or no active component in the supporting structures, the location of the active component is concentrated in the upper half of the individual microneedles in the array. In some embodiments, the active component concentrated in the upper half of the individual microneedles. The active component is concentrated in the tip of the microneedle, with the tip being defined by an area of the microneedle that extends from a base portion in a narrowing and/or tapered manner. The base portion, in turn, extends from the supporting structure of the array.

As noted above, in some embodiments, individual microneedles can comprise active components only in the upper half of the microneedle. In other embodiments, individual microneedles can comprise active components only in the tips or in a narrowing portion near the tip of the microneedle. In still other embodiments. individual needles can comprise active components throughout the entire microneedle portion that extends from the supporting structure, see U.S. Published Patent Application No. US-2016-0271381-A1, which is incorporated herein by reference.

The disclosed tolerogenic compositions can also be delivered as disclosed in PCT Application No. PCT/US2016/057363, which is incorporated herein by reference. This PCT application disclosed microneedle arrays that can be configured to penetrate the stratum corneum to deliver their cargo (e.g., biologics or bioactive components) to the epidermis and/or dermis, while minimizing pain and bleeding by preventing penetration to deeper layers that may contain nerve endings and vessels. Pyramidal CMC-microneedles effectively penetrated the stratum corneum, epidermis, and dermis of living human skin. and thus can be used for cutaneous delivery. Thus, in some embodiments, the microneedle array includes pyramidal CMC-microneedles. In other embodiments, the microneedle array includes obelisk shaped needles.

To construct the microneedle arrays, a base material can be used to form portions of each microneedle that have bioactive components and portions that do not. As discussed above, each microneedle can comprise bioactive components only in the microneedles, or in some embodiments, only in the upper half of the microneedles, or in other embodiments, only in a portion of the microneedle that tapers near the tip. Thus, to control the delivery of the bioactive component(s) and to control the cost of the microneedle arrays, each microneedle can have a portion with a bioactive component (immunogen and/or adjuvant) and a portion without a bioactive component. In the embodiments described herein, the portion without the bioactive component includes the supporting structure of the microneedle array and, in some embodiments, a base portion (e.g., a lower half) of each microneedle in the array.

In some embodiments, the dissolvable microneedle array includes wherein the dissolvable microneedle array comprises: i) a substrate comprising a biocompatible material that forms base portion; and ii) a plurality of microneedles extending from the base portion. Various biocompatible materials can be used as the base material for the microneedle arrays.

One bioactive components, such as the CRISPR-Cas effector polypeptides or a fragment thereof, or a fusion polypeptides comprising a CRISPR-Cas effector polypeptide and/or a vitamin D or an analog thereof can be covalently bonded to the biocompatible material, such as by a disulfide bond. In some cases, the biocompatible material can be carboxymethylcellulose. The linkages can be cleavable in vivo by an enzyme, and/or in response to pH, temperature, or both. The one or more bioactive components can be the same or different bioactive components, and one, or both, of the bioactive components can be conjugated to the biocompatible material. Bioactive components can include, but are not limited to, agents that promote differentiation of tolerogenic DCs, CRISPR-Cas effector polypeptides, an immunogenic fragment thereof, or a fusion polypeptide.

The structural substrates of biodegradable solid microneedles most commonly include poly(lactic-co-glycolic acid) (PLGA) or carboxymethylcellulose (CMC) based formulations; however, other bases can be used. Other polymeric materials, include polyvinylpyrrolidone (PVP), polyvinyl alcohol (PVA), hyaluronic acid (HA), gelatin, and/or mixtures of two or more of these. Other small molecule excipients, such as nonreducing sugars (e.g., trehalose, sucrose, etc), can be included. PLGA is a biodegradable polymer that is NOT water soluble, while CMC, PVP, PVA, HA, and gelatin are dissolvable in water.

In some embodiments, the biocompatible material is CMC. PLGA based devices can limit drug delivery and protein delivery applications due to the relatively high temperature (e.g., 135 degrees Celsius or higher) and vacuum and/or organic solvents required for fabrication. In contrast, a CMC-based matrix can be formed at room temperature in a simple spin-casting and drying process, making CMC-microneedle arrays more desirable for incorporation of a CRISPR-Cas effector polypeptide or immunogenic fragment thereof, or a fusion polypeptide comprising a CRISPR-Cas effector polypeptide or immunogenic fragment thereof.

CMC-hydrogel can be prepared from low viscosity sodium salt of CMC with or without active components (as described below) in sterile dH₂O. In the exemplary embodiment, CMC can be mixed with sterile distilled water (dH₂O) and with the active components to achieve about 25 wt % CMC concentration. The resulting mixture can be stirred to homogeneity and equilibrated at about 4 degrees Celsius for 24 hours. During this period, the CMC and any other components can be hydrated and a hydrogel can be formed. The hydrogel can be degassed in a vacuum for about an hour and centrifuged at about 20,000 g for an hour to remove residual micro-sized air bubbles that might interfere with a spincasting/drying process of the CMC-microneedle arrays. The dry matter content of the hydrogel can be tested by drying a fraction (10g) of it at 85 degrees Celsius for about 72 hours. The ready-to-use CMC-hydrogel is desirably stored at about 4 degrees Celsius until use.

Active components, such as a CRISPR-Cas effector polypeptide, immunogenic fragment thereof, or a fusion polypeptide comprising a CRISPR-Cas effector polypeptide or immunogenic fragment thereof, and optionally another agent, such as vitamin D or a vitamin D analog, can be incorporated in a hydrogel of CMC at a relatively high (20-30%) CMC-dry biologics weight ratio before the spin-casting process. Arrays can be spin-cast at room temperature, making the process compatible with the functional stability of a structurally broad range of bioactive components. Since the master and production molds can be reusable for a large number of fabrication cycles, the fabrication costs can be greatly reduced. The resulting dehydrated CMC-microneedle arrays are generally stable at room temperature or slightly lower temperatures (such as about 4 degrees Celsius), and preserve the activity of the incorporated biologics, facilitating easy, low cost storage and distribution.

In an exemplary embodiment, the surface of the production molds can be covered with about 50 μl (for molds with 11 mm diameter) of CMC-hydrogel and spin-casted by centrifugation at 2,500 g for about 5 minutes. After the initial CMC-hydrogel layer, another 50 μl CMC-hydrogel can be layered over the mold and centrifuged for about 4 hours at 2,500 g. At the end of a drying process, the CMC-microneedle arrays can be separated from the molds, trimmed off from excess material at the edges, collected and stored at about 4 degrees Celsius. The production molds can be cleaned and reused for further casting of microneedle arrays.

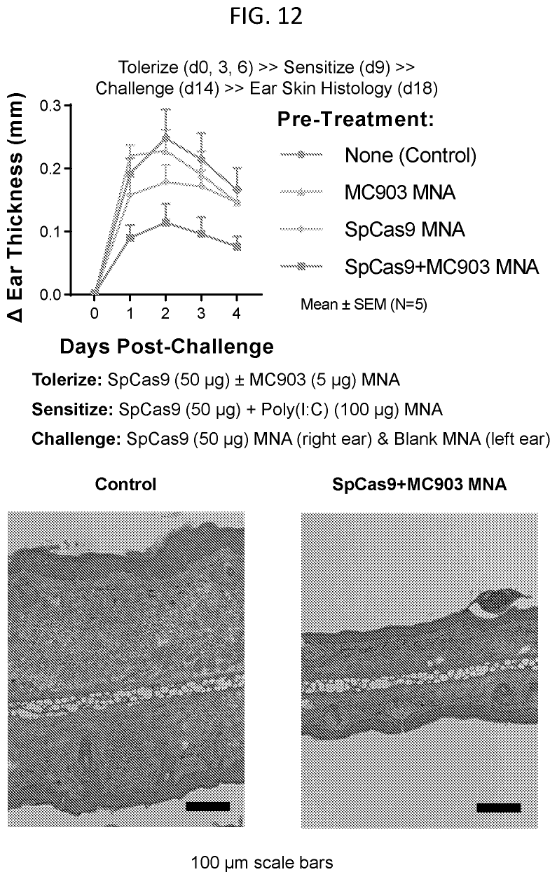

In some embodiments, CMC-solids can be formed with layers that do not contain active components and layers that contain active components. FIGS. 11A-D of PCT Application No. PCT/US2016/057363, incorporated herein by reference) illustrate CMC-solids with different shapes (FIG. 11A and 11B of PCT Application No. PCT/US2016/057363) and embedded active cargos on an upper layer which becomes, after micromilling, the portions of the microneedle with the active components. FIGS. 12A and 12B of PCT/US2016/057363, also illustrate CMC-solids with different shapes, with FIG. 12B of PCT/US2016/057363 showing a square shape and FIG. 12B showing a rectangular shape. Both CMC solids can be milled to dimensions for further processing as described herein. It should be understood that the geometries are not intended to be limiting. Any geometry can be used in the methods disclosed herein.

Generally, the microneedles have the mechanical strength to remain intact for delivery, while being inserted into the skin, while remaining in place for up to a number of days, and while being removed. The microneedles can have straight or tapered shafts. In a one embodiment, the diameter of the microneedle is greatest at the base end of the microneedle and tapers to a point at the end distal the base. The microneedle can also be fabricated to have a shaft that includes both a straight (untapered) portion and a tapered portion.

The microneedles can be formed with shafts that have a circular cross-section in the perpendicular, or the cross-section can be non-circular. For example, the cross-section of the microneedle can be polygonal (e.g., star-shaped, square, triangular), oblong, or another shape. The shaft can have one or more bores. The cross-sectional dimensions typically are between about 10 nm and 1 mm, such as between 1 μm and 200 μm, and more preferably between 10 μm and 100 μm. The outer diameter is typically between about 10 μm and about 100 μm and the inner diameter is typically between about 3 μm and about 80 μm.

The length of the microneedles typically is between about 1um and 1 mm, such as between about 1 μm and 50 μm, between about 50 μm and 100 μm, between about 100 μm and 250 μm, between about 250 μm and 500 μm, between about 500 μm and 750 μm, between about 750 μm and 850 μm, or between about 750 μm and 1 mm. The length is selected for the particular application, accounting for both an inserted and uninserted portion. An array of microneedles can include a mixture of microneedles having, for example, various lengths, outer diameters, inner diameters, cross-sectional shapes, and spacings between the microneedles.

The microneedles can be oriented perpendicular or at an angle to the supporting structure of the microneedle array. Preferably, the microneedles are oriented perpendicular to supporting structure so that a larger density of microneedles per unit area of substrate can be provided. An array of microneedles can include a mixture of microneedle orientations, heights, or other parameters.

In some cases, the supporting structure of the microneedle array is a substrate with an area typically between about 50 mm² and 150 mm², such as between about 100 mm² and 150 mm², between about 75 mm² and 100 mm², between about 50 mm² and 75 mm², between about 50 mm² and 65 mm², or between about 60 mm² and 65 mm².

In some cases, the substrate and/or microneedles, as well as other components, are formed from flexible materials to allow the device to fit the contours of the biological barrier, such as the skin, vessel walls, or the eye, to which the device is applied. A flexible device can facilitate more consistent penetration during use, since penetration can be limited by deviations in the attachment surface. For example, the surface of human skin is not flat due to dermatoglyphics (i.e. tiny wrinkles) and hair.

In some cases, a tolerogenic composition comprising a microneedle array is administered intradermally. In some cases, a tolerogenic composition comprising a microneedle array is administered subdermally. In some cases, a tolerogenic composition comprising a microneedle array is administered subcutaneously.

### Methods for Inducing Tolerance

The present disclosure provides methods of inducing tolerance to a CRISPR-Cas effector polypeptide in a mammalian subject, the method comprising administering to the subject an effective amount of a tolerogenic composition of the present disclosure. In some embodiments, the tolerogenic composition includes: a) one or more microparticles; b) one or more regulatory Treg stimulatory agents encapsulated within each microparticle; and c) a CRISPR-Cas effector polypeptide or an immunogenic fragment thereof, or a fusion polypeptide comprising a CRISPR-Cas effector polypeptide or immunogenic fragment thereof. In other embodiments, the tolerogenic composition includes: a) a dissolvable microneedle array; b) a vitamin D analog or other agent; and c) a CRISPR-Cas effector polypeptide or an immunogenic fragment thereof, or a fusion polypeptide comprising a CRISPR-Cas effector polypeptide or immunogenic fragment thereof. The methods generally involve administering to a subject in need thereof an effective amount of a tolerogenic composition of the present disclosure.

In some cases, a method of the present disclosure induces immunological tolerance to a CRISPR-Cas polypeptide in a subject, and thereby reduces the reactive immunological response following administration of the CRISPR-Cas polypeptide to the subject. Thus, the present disclosure provides methods of reducing the immune response to a CRISPR-Cas polypeptide in a subject, the method comprising administering to the subject an effective amount of a tolerogenic composition.

Subjects suitable for treatment with a method of the present disclosure include subjects requiring gene therapy or subjects who will be administered a gene-editing composition, such as to treat a disease or as an antiviral, antipathogenic, or anticancer therapeutic, or for biological research. The subject may be a neonate, a juvenile, or an adult. Of particular interest are mammalian subjects. Mammalian species that may be treated with the present methods include canines and felines; equines; bovines; ovines; etc, and primates, particularly humans. Animal models, particularly small mammals (e.g. mouse, rat, guinea pig, hamster, lagomorpha (e.g., rabbit), etc.) may be used for experimental investigations. In some non-limiting examples, the subject is human. In other non-limiting examples, the subject is a veterinary subject, including non-human primates. In some embodiments, the subject can have previously had a gene editing procedure, such as with the same or a different CRISPR-Cas effector polypeptide or an immunogenic fragment thereof, or a fusion polypeptide comprising a CRISPR-Cas effector polypeptide or immunogenic fragment thereof. These subjects can be selected for treatment.

In some embodiments, the effective of the tolerogenic composition, in one or more doses, stimulates Tregs in a subject such as by inducing Tregs or recruiting Tregs. In some cases, the effective tolerogenic composition, in one or more doses, increases the number of Tregs in a subject. CD4⁺, FOXP3⁺, and CD25⁺ Tregs and/or CD4⁺FoxP3⁻IL-10⁺Tr1 cells can suppress reactive T cells. The effective tolerogenic composition, in one or more doses, can act on naïve T cells to include differentiation to Tregs. In some cases, an effective amount of a tolerogenic composition is an amount that, when administered to a subject in need thereof in one or more doses, increases the number of Tregs in the subject by at least 10%, at least 15%, at least 20%, at least 25%, at least 30%, at least 40%, at least 50%, at least 60%, at least 70%, at least 80%, at least 90%, at least 2-fold, at least 2.5-fold, or more than 2.5-fold, compared to the number of Tregs in the subject before treatment with the tolerogenic composition as determined using an assay described herein or others known to one of skill in the art.

In some cases, the effective amount of a tolerogenic composition, in one or more doses, increases the number of tolerogenic DCs in the subject. In some cases, an effective amount of a tolerogenic composition is an amount that, when administered to a subject in need thereof in one or more doses, increases the number of tolerogenic DCs in the subject by at least 10%, at least 15%, at least 20%, at least 25%, at least 30%, at least 40%, at least 50%, at least 60%, at least 70%, at least 80%, at least 90%, at least 2-fold, at least 2.5-fold, or more than 2.5-fold, compared to the number of tolerogenic DCs in the subject before treatment with the tolerogenic composition.

In some cases, the effective amount of a tolerogenic composition, in one or more doses, increases the immune tolerance induction capability of tolerogenic DCs. The immune tolerance induction capability of tolerogenic DCs can be assessed using techniques known to one skilled in the art. Various assays known in the art can be used to assess whether tolerogenic DCs described herein induce immune tolerance. In one aspect, tolerogenic DCs described herein induce immune tolerance by creating an anti-inflammatory environment through the increased secretion anti-inflammatory of cytokines (e.g. IL-10) and attenuated secretion of pro-inflammatory cytokines (e.g. IL-12p70, IL-6, TNFα). In some cases, the ability of tolerogenic DCs to secrete IL-10, IL-12p70, IL-6 and TNFα is assessed using ELISA assays or Luminex xMAP assays. In some cases, the effective amount of a tolerogenic composition, in one or more doses, increases the immune tolerance induction capability of tolerogenic DCs in the subject by at least 10%, at least 15%, at least 20%, at least 25%, at least 30%, at least 40%, at least 50%, at least 60%, at least 70%, at least 80%, at least 90%, at least 2-fold, at least 2.5-fold, or more than 2.5-fold, compared to the immune tolerance induction capability of tolerogenic DCs in the subject before treatment with the tolerogenic composition.

In some embodiments, the tolerogenic composition, when administered in one or more doses to a subject in need thereof, ameliorates one or more effects associated with the immunological response to a CRISPR-Cas effector polypeptide in the subject. In some instances, the tolerogenic composition reduces the number of CD4⁺ reactive T cells (i.e., the number of CD4⁺ T cells reactive with the CRISPR-Cas effector polypeptide), which in turn leads to a reduction in CD8⁺ reactive with the CRISPR-Cas effector polypeptide. In some instances, the tolerogenic composition increases the number and/or activity of CD4⁺ Tregs, which in turn reduces the number and/or activity of CD4⁺ reactive T cells and/or CD8⁺ T reactive T cells. These cells can be measured in a sample from the subject.

Methods for determining the immunological response to a CRISPR-Cas effector polypeptide are well known in the art. In some cases, the immunological response is determined by measuring the level of T-cell activation induced by CRISPR-Cas effector polypeptides. In some embodiments, the T-cells comprise CD4⁺ and/or CD8⁺ T-cells. In certain embodiments, the level of activation of T-cells is measured using methods well-known in the art, including, but not limited to, flow cytometry, intracellular cytokine staining (ICS), staining of a degranulation marker, and immunohistochemical staining. In certain embodiments, markers for T cell activation include, but are not limited to, CD137 and/or CD154 and/or CD107a (a degranulation marker). In certain embodiments, activated T cells can be identified by the production of cytokines such as IFN-γ, tumor necrosis factor-α (TNF-α), and interleukin-2 (IL-2). In some cases, the immunological response is measured by detecting the presence of Cas-specific antibodies by ELISA or other assays known in the art. The immunological response can also be evaluated by determining the number of Treg cells, such as by fluorescence activated cell sorting to detect expression of CD4, CD25 and/or FoxP3.

In some embodiments, the tolerogenic composition of the present disclosure is administered to the subject prior to administering to a subject a gene-editing composition comprising a CRISPR-Cas effector polypeptide. The tolerogenic composition can be administered between about one day and about nine months before the gene-editing composition, such as between about one day and about one week, between about one week and about three weeks, between about three weeks and about one month. between about one month and about two months, between about two months and about four months, between about four months and about six months, or between about six months and about nine months. In an embodiment, the gene-editing composition is administered to the subject within about six months of administration of the tolerogenic composition.

In some cases, the method includes administering to the subject a gene-editing composition comprising a CRISPR-Cas effector polypeptide before administering the tolerogenic composition of the present disclosure. The gene-editing composition can be administered between one day and nine months before the tolerogenic composition, such as between one day and one week, between one week and three weeks, between three weeks and one month, between one month and two months, between two months and four months, between four months and six months, or between six months and nine months before the tolerogenic composition. In some cases, the gene-editing composition is administered to the subject within six months of administration of the tolerogenic composition. The gene-editing composition can include the same the CRISPR-Cas effector polypeptide, or a different, but related, the CRISPR-Cas effector polypeptide. In some embodiments, the subject has previously been administered a gene editing composition, prior to administering the tolerogenic composition, and then is subsequently administered another gene editing composition following administration of the tolerogenic composition. Thus, in this embodiment, the subject undergoes more than one gene editing procedure.

In some embodiments, the method includes administering to the subject a gene-editing composition comprising a CRISPR-Cas effector polypeptide in addition to the tolerogenic composition of the present disclosure, wherein the tolerogenic composition induces tolerance to the CRISPR-Cas effector polypeptide present in the gene-editing composition. The administration can be simultaneous.

In these embodiments, the gene-editing composition can include, but is not limited to, guide RNA (gRNA) and the CRISPR-Cas effector polypeptide. CRISPR-Cas effector polypeptides suitable for the gene-editing composition include, but are not limited to, type II CRISPR-Cas polypeptides, type V CRISPR-Cas polypeptides, type VI CRISPR-Cas polypeptides, CRISPRi polypeptides, CRISPRa polypeptides, CRISPRoff polypeptides, and other CRISPR-Cas polypeptides modified as required for the gene-editing composition. In some cases, the CRISPR-Cas effector polypeptide suitable for inclusion in a gene-editing composition includes a catalytically inactive CRISPR-Cas effector polypeptide that retains binding (when complexed with a guide RNA) to a target nucleic acid. In some cases, the gene-editing composition comprises gRNA or a nucleic acid encoding the guide RNA. In some cases, the gene-editing composition comprises a nucleic acid encoding a sequence to be inserted into the target nucleic acid.

A suitable dosage of a disclosed tolerogenic composition can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. The repetition rates for dosing can be determined based on measured residence times and concentrations of the administered agent in bodily fluids or tissues. Following successful treatment, it may be desirable to have the patient undergo maintenance therapy, wherein a tolerogenic composition of the present disclosure is administered in maintenance doses for subsequent gene therapy procedures. Those of skill will readily appreciate that dose levels can vary as a function of the type of the tolerogenic composition, the route of administration, and the susceptibility of the subject to side effects.

In some cases, multiple doses of a tolerogenic composition of the present disclosure are administered, such that the tolerogenic composition is administered to the same subject repeatedly. In some embodiments, at least two doses are administered as a prime and boost. The prime and boost can be the same dose, or different doses.

The frequency of administration of a tolerogenic composition of the present disclosure can vary depending on any of a variety of factors, e.g., severity of the symptoms, etc. For example, in some cases, a tolerogenic composition of the present disclosure is administered once per month, twice per month, three times per month, every other week (qow), once per week (qw), once every two weeks, once every three weeks, once every four weeks, twice per week (biw), three times per week (tiw), four times per week, five times per week, six times per week, every other day (qod), daily (qd), twice a day (qid), or three times a day (tid). Where the tolerogenic composition of the present disclosure is administered intravenously, administration once every week, once every two weeks, once every three weeks or once every four weeks or once every month may be commonly employed at the beginning of treatment. The tolerogenic composition can be administered in the tolerogenic composition is administered in a prime-boost strategy to the subject, wherein full tolerance is induced following the second (boost) administration.

The duration of administration of a tolerogenic composition of the present disclosure, e.g., the period of time over which a tolerogenic composition of the present disclosure is administered, can vary, depending on any of a variety of factors, e.g., patient response, etc. For example, a tolerogenic composition of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. A tolerogenic composition of the present disclosure is administered to a subject using any available method and route suitable for drug delivery, including in vivo and in vitro methods, as well as systemic and localized routes of administration. Suitable administration methods are listed above.

### Kits

Kits are provided herein that include a tolerogenic composition. The tolerogenic composition can include a1) a microparticle; b1) one or more Treg stimulating agents encapsulated within the microparticle; and c1) a CRISPR-Cas effector polypeptide or immunogenic fragment thereof, or a fusion polypeptide comprising a CRISPR-Cas effector polypeptide or immunogenic fragment thereof. The kit can include multiple types of microparticles, each encapsulating a different Treg stimulating agent, or one type of microparticle encapsulating more than one Treg stimulating agent.

The tolerogenic composition can include a2) a dissolvable microneedle array; b2) one or more agents that promote differentiation of tolerogenic DCs; or c2) a CRISPR-Cas effector polypeptide or immunogenic fragment thereof, or a fusion polypeptide comprising a CRISPR-Cas effector polypeptide or immunogenic fragment thereof.

The kits may also include additional components to facilitate the particular application for which the kit is designed. For example, kits may additionally include buffers and other reagents routinely used for the practice of a particular method. The kit can include a gene-editing composition comprising the CRISPR-Cas effector polypeptide. The kit can include information that the tolerogenic composition induces tolerance to the CRISPR-Cas effector polypeptide present in the gene-editing composition. The gene editing system can include gRNAs for a gene of interest.

The kit can include a container and a label or package insert on or associated with the container. Suitable containers include, for example, bottles, vials, syringes, etc. The containers may be formed from a variety of materials such as glass or plastic. In several embodiments, the container may have an access port (for example the container may be an intravenous solution bag or a vial having a stopper pierceable by a hypodermic injection needle) so that a specific amount of an agent can be withdrawn.

In some embodiments, a label or package insert indicates the use of the composition(s), such as to induce tolerance. The package insert typically includes instructions customarily included in commercial packages of products that contain information about the indications, dosages, contraindications and/or warnings concerning the use of such products. The instructional materials may be written, in an electronic form (such as a computer diskette or compact disk) or may be visual (such as video files).

The disclosure is illustrated by the following non-limiting Examples.

### EXAMPLES

Gene editing using the CRISPR-Cas9 system has great potential for treatment of various human diseases. The Cas9 nucleases used to delete or insert genes are derived from bacteria to which humans are commonly exposed (e.g., S. pyogenes and S. aureus). As such, a large proportion of the population has pre-existing humoral and cellular immunity against these Cas9 proteins, which is a potential barrier for safe and effective in vivo gene editing (see Charlesworth et al., Nature Medicine, 2019). In particular, Cas9-specific memory T cells may attack and kill a patient's cells to which Cas9 was delivered or Cas9 expression induced. This could prevent successful gene editing and potentially cause tissue or organ toxicity.

Methods were developed to measure CD4 and CD8 T cell responses to Cas9 in mice, enabling evaluation of the approaches to reduce immunogenicity. Cas9 peptides were identified that were presented by MHC I and MHC II in a specific mouse strain. In addition, methods were used to induce immunological tolerance (non-responsiveness) to Cas9, see FIGS. 1A-1C. Two drug delivery systems that teach the immune system to tolerate Cas9, thereby potentially preventing deleterious immune responses against Cas9 that would preclude in vivo gene editing. The first approach uses injections of Cas9 protein together with regulatory T cell (Treg)-inducing microparticles (TRI MPs), biodegradable polymeric microparticles that provide sustained delivery of TGF-β1, rapamycin, and IL-2 to induce differentiation of naturally suppressive Tregs. The second approach uses co-delivery of Cas9 protein in a microneedle, such as with a tolerogenic vitamin D3 analog (MC903). Delivery to the skin microenvironment was achieved via dissolvable microneedle arrays (MNAs). The disclosed approaches ultimately promote increases in regulatory to inflammatory T-cell ratios, thereby preventing or reducing deleterious immune responses against cells that express or internalize Cas9 for the purposes of in vivo CRISPR gene therapy.

### Example 1

### Assays for Measuring Relevant Cas9 Responses in Mice

Two robust immunization regimens and ex-vivo restimulation protocols were established to measure SpCas9 specific T cell immunity in C57BL/6 mice. C57BL/6 immunocompetent mice were immunized with SpCas9 protein and adjuvant to elicit SpCas9 specific CD4 T cells or were injected with SpCas9 expressing cells to elicit SpCas9 specific CD8 T cells. Similar methods can be used to study immunity to other editors.

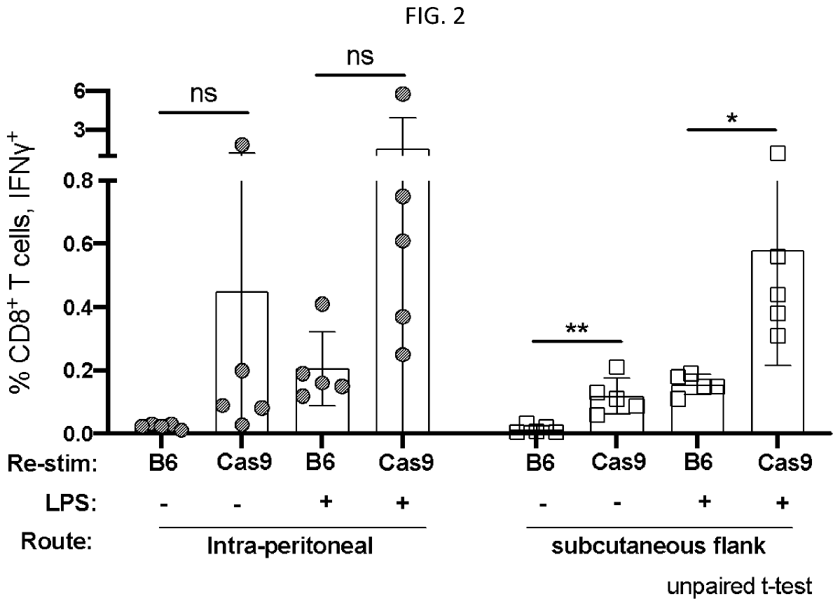

To induce SpCas9 specific CD8 T cell immunity, splenocytes were isolated that express SpCas9 intracellularly and were activated with 500 ng/ml lipopolysaccharide (LPS) overnight. These splenocytes were injected into naïve C57BL/6 mice twice (day 0 prime, day 14 boost). Splenocytes from immunized mice were re-stimulated in vitro with wildtype splenocytes or cells expressing Cas9. A statistically significant recall response to Cas9 was observed in animals immunized in the flank (FIG. 2).

To elicit SpCas9 specific CD4 T cells, C57BL/6 mice were immunized twice with SpCas9 protein formulated with TITERMAX™ Gold adjuvant (day 0 prime, day 14 boost). Splenocytes from immunized mice were harvested on day 21 and re-stimulated in-vitro with SpCas9 protein. Only mice immunized with SpCas9 displayed an antigen-specific recall response upon re-stimulation (FIG. 3).

### Example 2

### Two Different Methods of Tolerance Induction to Cas9

Results with tolerogenic microparticles. Mice were injected subcutaneously in the scruff of the neck with Cas9 co-delivered with microparticles (MP) containing rapamycin, TGF-beta, and IL-2 (Tri-MP). The microparticles provide a slow release of agents that induce an expansion of specific Tregs and ultimately provide systemic tolerance to an antigen-specific challenge. As shown in FIG. 4B, an expansion of regulatory T cells was observed in the spleen after administering Cas9 mixed with microparticles containing rapamycin, TGF-beta, and IL-2 (“Tri-MP Cas9”) but not when the rapamycin, TGF-beta, and IL-2 were excluded from the microparticles (“blank MP Cas9”), nor when Cas9 was excluded (“Tri-MP”).

To examine the impact on the immune response to Cas9, mice were pretreated with Tri-MP Cas9 and immunized 7 days later with Cas9 mixed with the TITERMAX® adjuvant. Seven days later, splenocytes from the mice were tested by Elispot assay for the presence of T cells producing IFN-g when stimulated with Cas9. Pretreatment with the Tri-MP Cas9 nearly completely suppressed the CD4 T cell response to Cas9 (FIG. 4A). Surprisingly a diminished response was also observed in mice pretreated with Tri-MP without Cas9, which may be due to the presence of residual immunosuppressive factors being present at the time of Cas9 challenge one week later. Hence, the procedures were repeated, delaying the immunization until 14 days after administering the MPs. The response was boosted with a second immunization one week later, and the spleen cells were tested one week after that using a cytokine bead array to quantify IFN-γ produced in the cultures (FIG. 4C). Pretreatments with TRI-MP mixed with Cas9 almost completely abolished the T cell responses. In contrast, pretreatments with TRI-MP alone or Blank-MP mixed with Cas9 had no statistically significant effect. These results show that co-delivery of Cas9 and TRI-MP can expand Tregs and eliminate Cas9 effector T cell responses.

Results with tolerogenic microneedles: A second method of tolerance induction involves applying tolerogenic microneedle arrays (MNAs) containing Cas9 or a control protein, BSA. Mice received three applications of MNAs. One week later they were immunized subcutaneously at a different site with Cas9 protein formulated with TITERMAX® adjuvant. One week later, splenocytes from the mice were harvested and T cell responses to Cas9 were evaluated by intracellular cytokine staining. MNA application, especially with Cas9, reduced responses to subsequent stimulation (FIG. 5). Surprisingly, the BSA control MNAs were nearly as effective as the Cas9 MNAs. Though unexpected, the results suggest that MNA application with Cas9 elicits an immunosuppressive environment that reduces subsequent immune responses to Cas9.

### Example 3

FIGS. 1A-1C show a proposed mechanism by which TRI MP and MC903 MNAs promote antigen-specific tolerance induction. For the specific Cas9 application, Cas9 antigen can be injected subcutaneously with TRI MPs rather than administered to the skin near the TRI MP injection site.

TRI MP+Cas9 can be administered one or more times before in vivo gene editing with the CRISPR-Cas9 system to reduce pre-existing immunity to Cas9. Alternatively, TRI MP can be administered together with the CRISPR-Cas9 in vivo gene editing system (i.e., at the time of editing), depending on the site of administration. Finally, TRI MP+Cas9 can be administered after a first round of gene editing, prior to subsequent round(s) of gene editing. Similarly. Cas9+MC903 MNAs can be administered one or more times prior to CRISPR-Cas9 gene editing to reduce pre-existing immunity to Cas9, and/or one or more times after a first round of gene editing, prior to subsequent round(s).

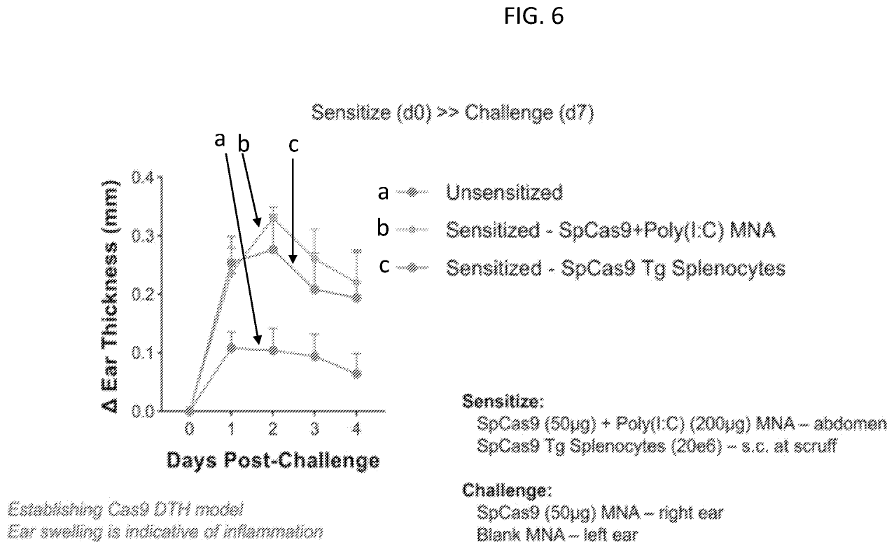

Mice were sensitized (immunized) to S. pyogenes Cas9 (SpCas9) antigen either by application of a Cas9 (antigen)+Poly(I:C) (adjuvant) MNA, or by subcutaneous injection of Cas9-expressing cells (specifically splenocytes). Seven days later, mice were challenged (re-exposed to Cas9 antigen) at the right ear via Cas9 MNA, and ear thickness was measured daily over the next four days. A blank (empty) MNA was applied to the contralateral ear to control for any increase in ear thickness not related to the Cas9 antigen-specific inflammatory response. Ear swelling associated with a delayed type hypersensitivity (T-cell mediated) response to Cas9 was reported as changes (delta) in ear thickness (i.e., Cas9 MNA treated right ear−Blank MNA treated left ear). Greater ear swelling is consistent with a stronger Cas9-specific, T-cell mediated immune response, see FIG. 6.

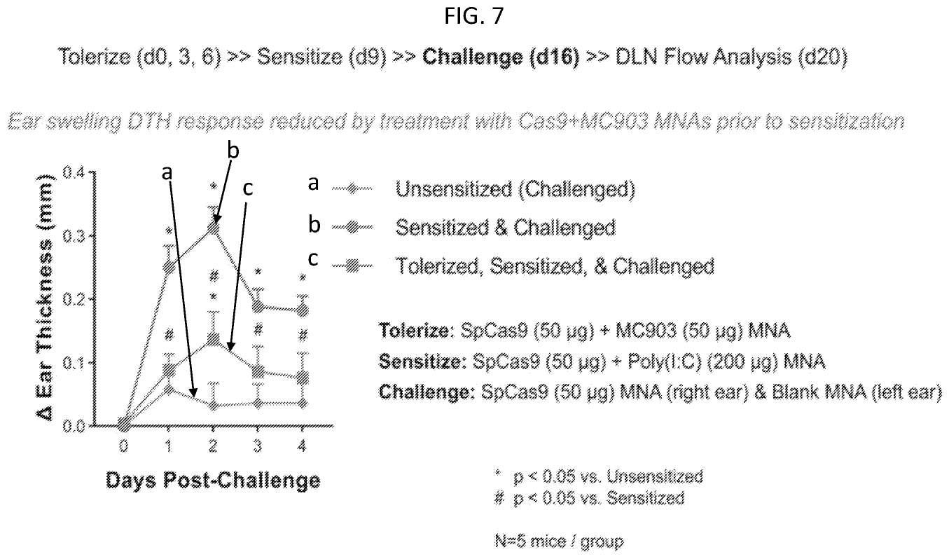

Prior to sensitization (immunization) with Cas9+PolyIC MNAs, mice were treated by three consecutive applications of Cas9+MC903 MNAs (days 0, 3, and 6). Three days later (day 9), mice were sensitized (immunized) to Cas9 antigen by application of a Cas9+Poly(I:C) MNA. Seven days later, mice were challenged (re-exposed to Cas9 antigen) at the right ear via Cas9 MNA, and ear thickness was measured daily over the next four days. A blank (empty) MNA was applied to the contralateral ear to control for any increase in ear thickness not related to the Cas9 antigen-specific inflammatory response. Ear swelling associated with a delayed type hypersensitivity (T-cell mediated) response to Cas9 was reported as changes (delta) in ear thickness (i.e., Cas9 MNA treated right ear−Blank MNA treated left ear). Greater ear swelling is consistent with a stronger Cas9-specific, T-cell mediated immune response, and pre-treatment with Cas9+MC903 MNAs (prophylactic tolerization) prevented/reduced subsequent sensitization, resulting in less of a delayed type hypersensitivity (ear swelling) response. Unsensitized mice (grey) did not have pre-existing immunity to Cas9 prior to ear challenge with Cas9 MNA. Results are shown in FIG. 7.

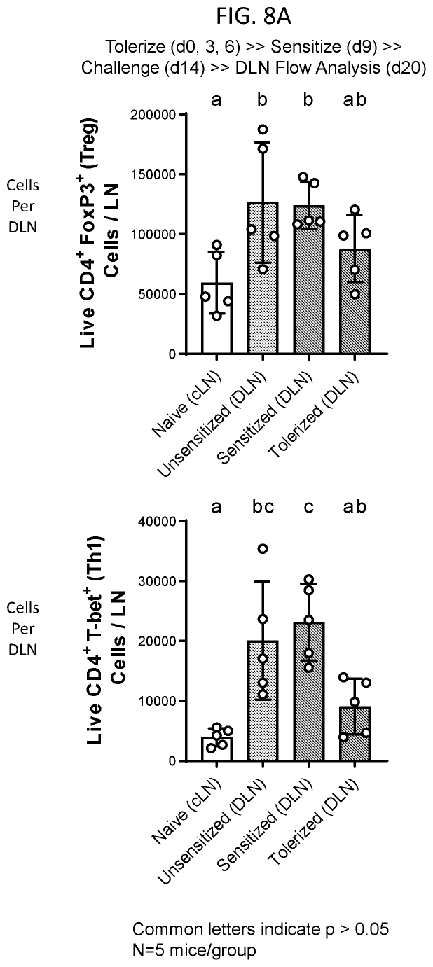

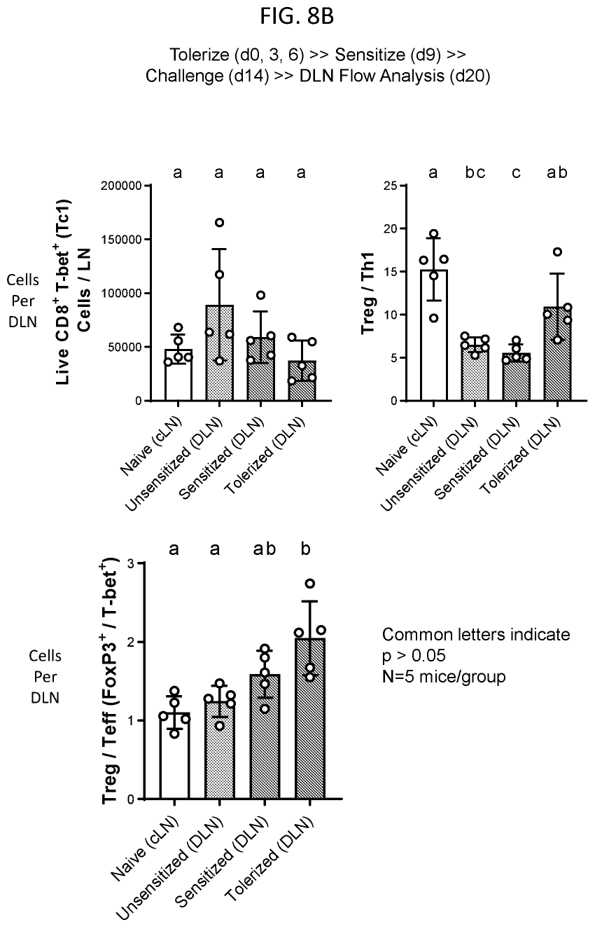

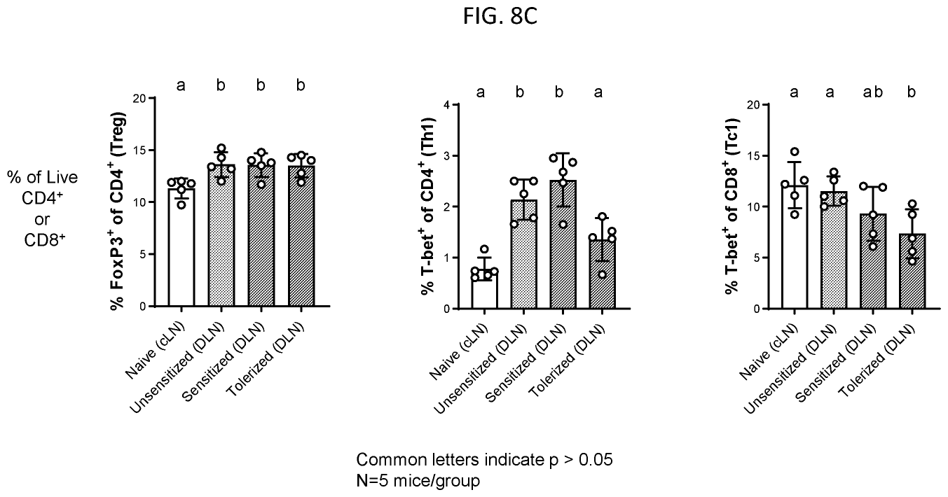

Four days after ear challenge (see FIG. 7) ear skin draining lymph nodes (DLN) were isolated, and T-cell responses evaluated by flow cytometry. In this model, Cas9+PolyIC MNA sensitization appear to induce a predominantly Th1-mediated pro-inflammatory effector T-cell response, and pre-treatment with Cas9+MC903 MNAs prior to sensitization significantly reduced Th1 populations and increased Treg/Th1 and Treg/Teff ratios, see FIGS. 8A-8C. This shift in the regulatory to effector T-cell ratio is consistent with induction of a more tolerogenic immune response.

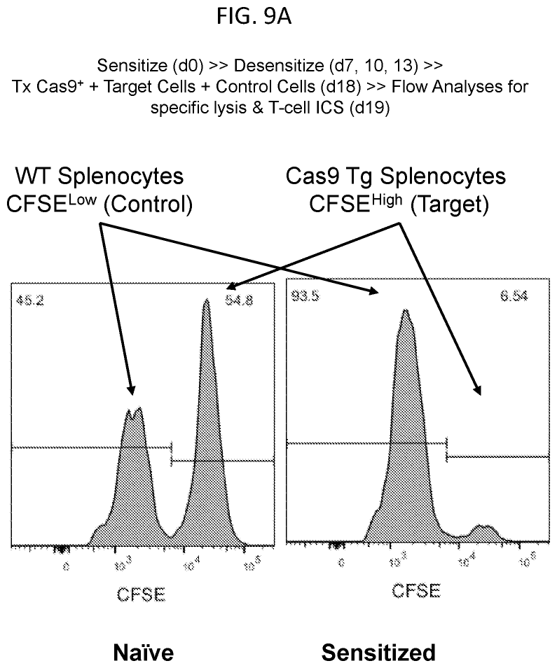

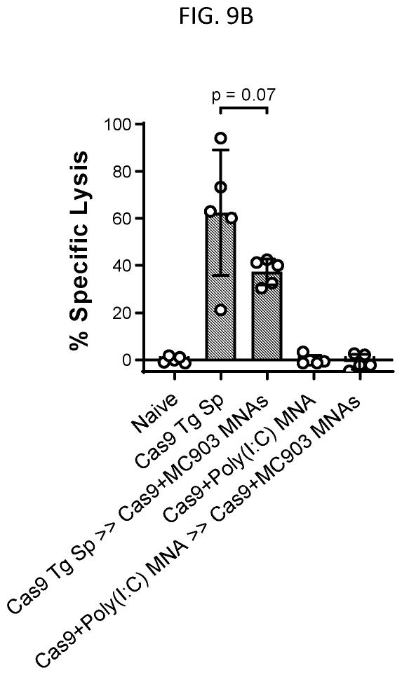

Unlike the previous prophylactic tolerance induction model, tolerance was induced by treatment of previously sensitized mice (i.e., mice with pre-existing immunity to Cas9) with Cas9+MC903 MNAs, see FIGS. 9A-9B. In this therapeutic desensitization model, mice were first sensitized (immunized) to S. pyogenes Cas9 (SpCas9) antigen either by application of a Cas9+Poly(I:C) MNA, or by subcutaneous injection of Cas9-expressing splenocytes. Starting one week later, some mice were treated with Cas9+MC903 MNAs (days 7, 10, and 13). Five days after the last treatment with Cas9+MC903 MNAs (day 18), all mice received an adoptive transfer of a mix of Cas9-expressing CFSE^{high} “target” cells (splenocytes) and “control” CFSE^{low} cells (i.e., splenocytes that don't express Cas9). Target cells were labeled with CFSE dye (10 uM) and control cells were labeled with CFSE (1 uM), so they could be identified and discriminated by flow cytometry. The next day, spleens of mice were processed, and analyzed by flow cytometry to measure specific cell lysis (i.e., specific killing of Cas9-expressing target cells by Cas9-specific effector T cells, especially cytotoxic T cells). Percent specific lysis is calculated as {1-[(mean CFSE^{low}/CFSE^{high} ratio from naïve mice)/(CFSE^{low}/CFSE^{high} ratio from immunized mouse)]}×100%.

As expected, naïve mice exhibit no specific lysis of Cas9-expressing target cells, consistent with a lack of Cas9-specific cytotoxic T cells. Mice sensitized with Cas9+PolyIC MNAs also did not exhibit specific lysis, suggesting that this sensitization method did not induce a robust cytotoxic T-cell response. This is consistent with the results in FIGS. 8A-8C showing a predominant Th1 response, and may result from a lack of cross-presentation of exogenous (extracellular protein) antigen to CD8+ T cells. In contrast, sensitization with Cas9-expressing splenocytes injected subcutaneously resulted in specific lysis of Cas9-expressing target cells (see FIGS. 9A-9B), and the degree of specific lysis was somewhat reduced by treatment with Cas9+MC903 MNAs after sensitization (see FIGS. 9A-9B). This in vivo specific lytic assay is a relevant indicator of tolerance induction and suppression of Cas9-specific T-cell responses, since preventing the lysis or killing of Cas9-expressing cells by the immune system is the ultimate goal to enable in vivo editing with CRISPR-Cas9.

Pretreatment with SpCas9 and Mc903 MNAs reduced sensitization and subsequent delayed type hypersensitivity response. C57BL/6 mice were treated with MC903 MNAs, SpCas9 MNAs, or SpCas9+MC903 MNAs (days 0, 3, and 6) prior to sensitization with SpCas9+PolyIC MNAs (day 9). Control mice were sensitized but not tolerized (pre-treated). Five days after sensitization, a DTH response was elicited by application of SpCas9 MNA to the right ears. Blank MNAs were applied to the contralateral ears, and ear swelling one to four days post-challenge was reported as the difference between ear thickness of the SpCas9 MNA treated ears and blank MNA treated ears. Pre-treatment with SpCas9+MC903 MNAs reduced the ear swelling DTH response to SpCas9 challenge, see FIG. 12. In contrast, pre-treatment with MC903 MNAs or SpCas9 MNAs had minimal effect on ear swelling responses, suggesting co-delivery of SpCas9 plus MC903 via MNAs was necessary to reduce ear swelling (evidencing reduction of antigen-specific, T-cell mediated inflammation).

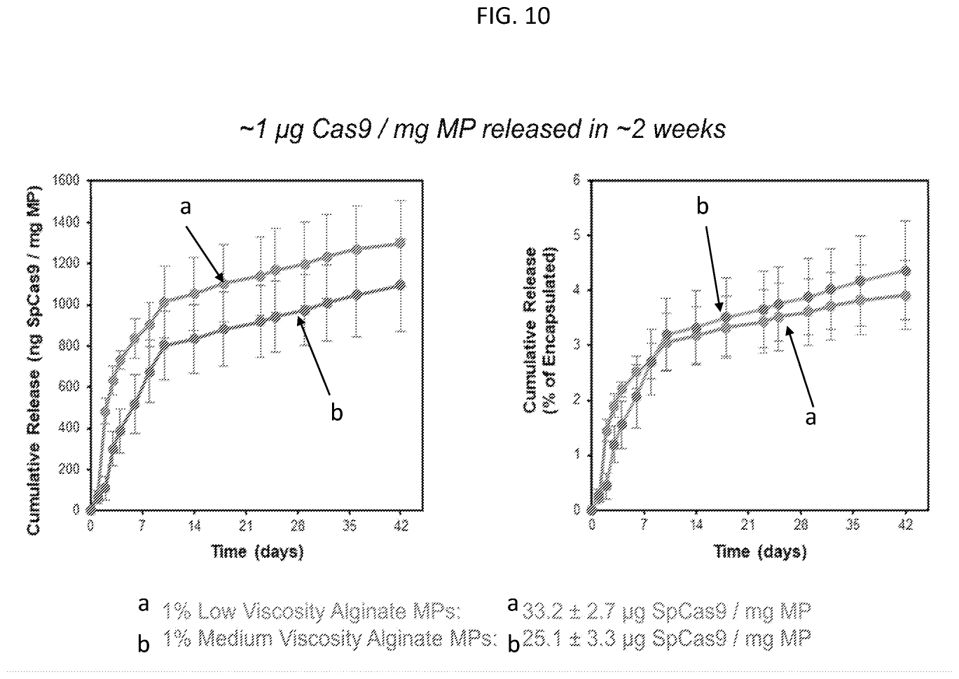

FIG. 10 shows that sustained release of S. pyogenes (SpCas9) protein can be achieved by encapsulation in alginate hydrogel microparticles (MPs). Cas9 was loaded in low viscosity or medium viscosity alginate MPs that were cross-linked with CaCl₂. In vitro release assays were conducted by incubating a known mass of Cas9 MPs in PBS+1% BSA buffer at 37C. At various time points, suspensions of MPs were centrifuged, supernatants containing released Cas9 were sampled, and MPs were resuspended in fresh buffer. Cas9 concentrations in release buffer were measured by ELISA, and cumulative release was calculated in terms of amount of Cas9 released per mass of MPs. In FIG. 10, the left graph shows total cumulative release (ng Cas9/mg MP), and the right shows cumulative release as a percentage of the total Cas9 encapsulated.

It will be apparent that the precise details of the methods or compositions described may be varied or modified without departing from the spirit of the described invention. We claim all such modifications and variations that fall within the scope and spirit of the claims below.

1. A tolerogenic composition comprising:
a1) one or more microparticles;
b1) one or more regulatory T cell (Treg) stimulating agents encapsulated within the one or more microparticles; and
c1) a CRISPR-Cas effector polypeptide or immunogenic fragment thereof, or a fusion polypeptide comprising a CRISPR-Cas effector polypeptide or an immunogenic fragment thereof; or
a2) a dissolvable microneedle array;
b2) one or more agents that promote differentiation of tolerogenic DCs in the dissolvable microneedle array; and
c2) a CRISPR-Cas effector polypeptide or immunogenic fragment thereof, or a fusion polypeptide comprising a CRISPR-Cas effector polypeptide or an immunogenic fragment thereof.

2. The tolerogenic composition of claim 1, comprising:
a1) the one or more microparticles;
b1) the one or more regulatory T cell (Treg) stimulating agents encapsulated within the one or more microparticles; and
c1) the CRISPR-Cas effector polypeptide or immunogenic fragment thereof, or a fusion polypeptide comprising a CRISPR-Cas effector polypeptide.

3. The tolerogenic composition of claim 2, wherein the one or more Treg stimulating agents comprise CCL22, retinoic acid, or vasoactive intestinal peptide.

4. The tolerogenic composition of claim 2, wherein the one more Treg stimulating agents comprise IL-2, TGF-β, rapamycin, a rapamycin derivative, or a CCR8 ligand.

5. The tolerogenic composition of claim 2, wherein the one or more Treg stimulating agents comprise TGFβ, IL2, and rapamycin.

6. The tolerogenic composition of claim 2, wherein the tolerogenic composition comprises i) a microparticle comprising TGFβ; ii) a microparticle comprising IL-2, and iii) a microparticle comprising rapamycin.

7. The tolerogenic composition of claim 2, wherein the one or more microparticles comprises at least one polymer.

8. The tolerogenic composition of claim 7, wherein the at least one polymer comprises polyethylene glycol (PEG), a poly(amino acid), polylactate, polylactic acid, polyglutamic acid, polyglycolic acid (PGA), polylactic acid (PLA), poly(lactic-co-glycolic acid) (PLGA), polyvinyl acetate (PVA), poly(ethylene glycol-b-(DL-lactic acid-co-glycolic acid)-b-ethylene glycol) (PEG-PLGA-PEG), Poly (ethylene glycol)-b-poly(D,L-lactide-co-glycolide) (PEG-b-PLGA), polycaprolactone-PEG (PCL-PEG), poly(vinylidene fluoride)-PEG (PVDF-PEG), poly(lactic acid-co-PEG) (PLA-PEG), poly(methyl methacrylate)-PEG (PMMA-PEG), and combinations thereof.

9. The tolerogenic composition of claim 2, wherein the one or more microparticles comprise alginate.

10. The tolerogenic composition of claim 1, wherein one or more microparticles are formulated for sustained release of the one or more regulatory T cell (Treg) stimulating agents.

11. The tolerogenic composition of claim 1, wherein the CRISPR-Cas effector polypeptide is a type II CRISPR-Cas effector polypeptide, a type V CRISPR-Cas effector polypeptide, or a type VI CRISPR-Cas effector polypeptide.

12. The tolerogenic composition of claim 1, wherein the CRISPR-Cas effector fusion polypeptide comprises: i) a CRISPR-Cas effector polypeptide; and ii) one or more heterologous effector polypeptides.

13. The tolerogenic composition of claim 12, wherein at least one of the one or more heterologous effector polypeptides is a single stranded nuclease, a double strand nuclease, a helicase, a methylase, a demethylase, an acetylase, a deacetylase, a deaminase, an integrase, a recombinase, a base editor, or a prime editor.

14. A method of inducing tolerance to a CRISPR-Cas effector polypeptide in a mammalian subject, the method comprising administering to the subject an effective amount of a tolerogenic composition of claim 2, thereby inducing tolerance to the CRISPR-Cas effector polypeptide.

15. The method of claim 14, wherein the composition is administered intradermally, subdermally, subcutaneously, or intramuscularly.

16. The tolerogenic composition of claim 1, wherein the composition comprises:
a2) the dissolvable microneedle array;
b2) the one or more agents that promote differentiation of tolerogenic DCs in the dissolvable microneedle array;
c2) a CRISPR-Cas effector polypeptide or immunogenic fragment thereof, or a fusion polypeptide comprising a CRISPR-Cas effector polypeptide or the immunogenic fragment.

17. The tolerogenic composition of claim 16, wherein the dissolvable microneedle array comprises: i) a substrate comprising a biocompatible material that forms base portion; and ii) a plurality of microneedles extending from the base portion.

18. The tolerogenic composition of claim 17, wherein the biocompatible material comprises carboxymethylcellulose, polyvinylpyrrolidone (PVP), polyvinyl alcohol (PVA), hyaluronic acid (HA), or gelatin.

19. The tolerogenic composition of claim 18, wherein the biocompatible material comprises carboxymethylcellulose.

20. The tolerogenic composition of claim 16, wherein the one or more agents that promote differentiation of tolerogenic DCs comprise vitamin D3 or a vitamin D3 analog.

21. A method of inducing tolerance to a CRISPR-Cas effector polypeptide in a mammalian subject, comprising:
administering to the subject an effective amount of a tolerogenic composition of claim 17,
thereby inducing tolerance to the CRISPR-Cas effector polypeptide.

22. The method of claim 21, wherein the composition is administered intradermally or subdermally.

23. The method of claim 14, wherein the tolerogenic composition is administered repeatedly to the subject.

24. The method of claim 23, wherein the tolerogenic composition is administered in a prime-boost strategy to the subject.

25. The method of claim 14, comprising:
administering to the subject a gene-editing composition comprising the CRISPR-Cas effector polypeptide following administering the effective amount of the tolerogenic composition, wherein the tolerogenic composition induces tolerance to the CRISPR-Cas effector polypeptide present in the gene-editing composition.

26. The method of claim 25, wherein the gene-editing composition is administered to the subject within about 6 months of administration of the tolerogenic composition.

27. The method of claim 14, wherein the tolerogenic composition is administered to a subject that previously was administered a gene editing composition.

28. The method of claim 14, further comprising:
performing a gene editing procedure on the subject.

29. The method of claim 14, wherein the subject is a human.

30. The method of claim 1, wherein the subject is a non-human mammal.
